(19) United States
(12) Patent Application Publication
(10) Pub. No.: US 2013/0210779 A1
(43) Pub. Date: Aug. 15, 2013

(54) METHOD OF IDENTIFYING TRIPLE NEGATIVE BREAST CANCER

(71) Applicant: The Washington University, (US)

(72) Inventors: Susana Gonzalo, St. Louis, MO (US); Ignacio Gonzalez-Suarez, St. Louis, MO (US); David Grotsky, St. Louis, MO (US)

(73) Assignee: The Washington University, St. Louis, MO (US)

(21) Appl. No.: 13/768,577

(22) Filed: Feb. 15, 2013

Related U.S. Application Data
(60) Provisional application No. 61/599,241, filed on Feb. 15, 2012.

(51) Int. Cl. G01N 33/68; A61N 5/10

(52) U.S. Cl. CPC G01N 33/6893; A61N 5/10
USPC 514/167; 435/7.1; 435/7.92; 506/9; 435/375; 514/619; 514/248; 514/212.06; 514/394; 514/410; 600/1

(57) ABSTRACT

The present invention provides means for identifying or classifying breast tumors based on the levels of nuclear cathepsin-L (CTSL) and nuclear p53 binding protein (53BP1), and methods of treating thereof.

### CROSS REFERENCE TO RELATED APPLICATIONS

This application claims the priority of the U.S. provisional application No. 61/599,241, filed Feb. 15, 2012, which is hereby incorporated by reference in its entirety.

### FIELD OF THE INVENTION

The present invention provides means for identifying or classifying breast tumors based on the levels of nuclear cathepsin-L (CTSL) and nuclear p53 binding protein (53BP1), and methods of treatment thereof.

### BACKGROUND OF THE INVENTION

In breast cancers, current clinical management still relies on traditional prognostic and predictive factors including histologic, clinical and some well-defined biologic factors. Despite the overall association of these variables with prognosis and outcome, they are limited in their ability to capture the nuances of the complex cascade of events that drive the clinical behavior of breast cancer. Tumors of apparently homogenous morphologic characters still vary in response to therapy and have distinct outcomes.

The complexity and diversity of cancer phenotypes has fueled the concept of personalized treatment, which utilizes the molecular and genetic composition of the specific tumor to design the best therapeutic strategy. For personalized medicine to be effective, it is necessary to identify predictive biomarkers that allow the stratification of patients into different subgroups, and diagnostic tests that determine the clinical response of a patient subgroup to a specific drug.

Recent gene expression profiling of breast cancer has identified specific subtypes with clinical, biologic, and therapeutic implications. The basal-like group of tumors is characterized by an expression signature similar to that of the basal/myoepithelial cells of the breast and is reported to have transcriptomic characteristics similar to those of tumors arising in BRCA1 germline mutation carriers. They are associated with aggressive behavior and poor prognosis, and typically do not express hormone receptors or HER-2 (“triple-negative” phenotype). Therefore, patients with basal-like cancers are unlikely to benefit from currently available targeted systemic therapy. BRCA1/2 negative tumors are responsive to PARP inhibitors, such as olaparib and veliparib. However, not all tumors in these categories respond in the same manner to the chosen treatments.

Thus, there remains a need in the art for means to effectively classify a breast tumor, specifically basal-like tumors, BRCA1-deficient tumors, and triple-negative tumors, in a manner that provides clear treatment recommendations based on the classification.

### SUMMARY OF THE INVENTION

One aspect of the invention encompasses a method for classifying a breast tumor in a subject. The method comprises (a) obtaining a sample of a breast tumor from a subject, (b) processing the sample in vitro to determine the levels of nuclear cathepsin L and nuclear 53BP1 in at least one tumor cell comprising the sample, and (c) classifying the tumor as having (i) high nuclear cathepsin L and low nuclear 53BP1, (ii) high nuclear cathepsin L and high nuclear 53BP1, or (iii) low nuclear cathepsin L and low nuclear 53BP1.

Another aspect of the invention encompasses a method for increasing the sensitivity of a tumor cell to a DNA-damaging agent, the method comprising contacting the tumor cell with an effective amount of a cathepsin L inhibitor and a DNA-damaging agent.

Another aspect of the invention encompasses a method for predicting the effectiveness of a DNA damaging agent in reducing tumor growth in a subject in need thereof. The method comprises, (a) obtaining a sample of a breast tumor from the subject, (b) processing the sample in vitro to determine the levels of nuclear CTSL and nuclear 53BP1 in at least one tumor cell comprising the sample; (c) classifying the tumor as non-responsive to a DNA-damaging agent if the sample has (i) high nuclear CTSL and low nuclear 53BP1 or (ii) low nuclear CTSL and low nuclear 53BP1; and (d) identifying a subject with a tumor classified as non-responsive to a DNA-damaging agent as a subject for which a DNA-damaging agent would not be effective.

### REFERENCE TO COLOR FIGURES

This application files contains at least one photograph executed in color. Copies of this patent application publication with color photographs will be provided by the Office upon request and payment of the necessary fee.

### BRIEF DESCRIPTION OF THE FIGURES

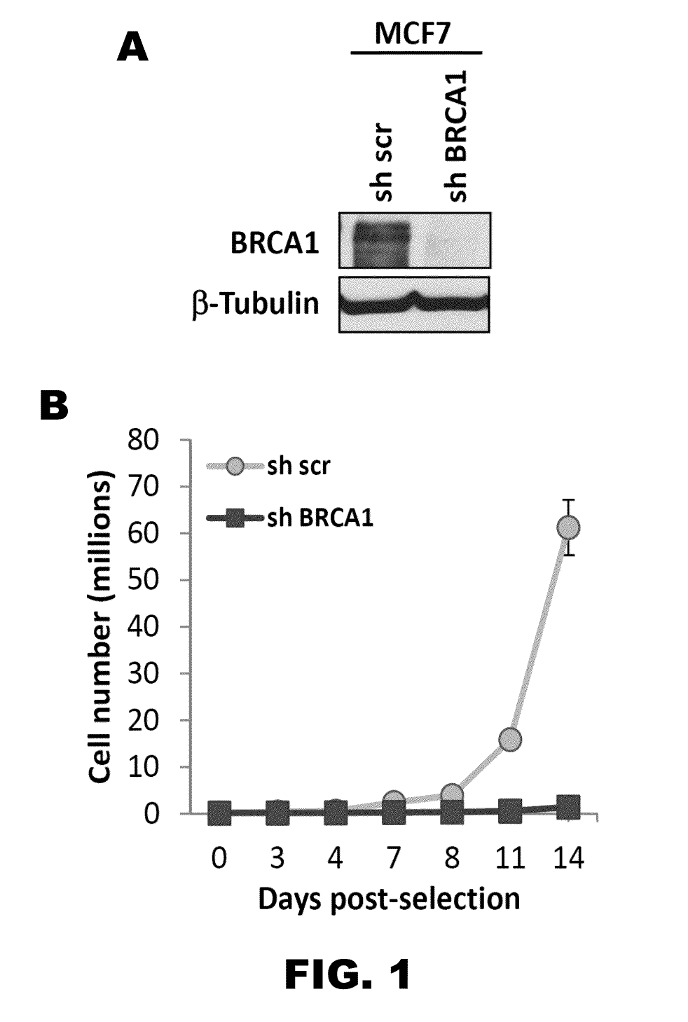

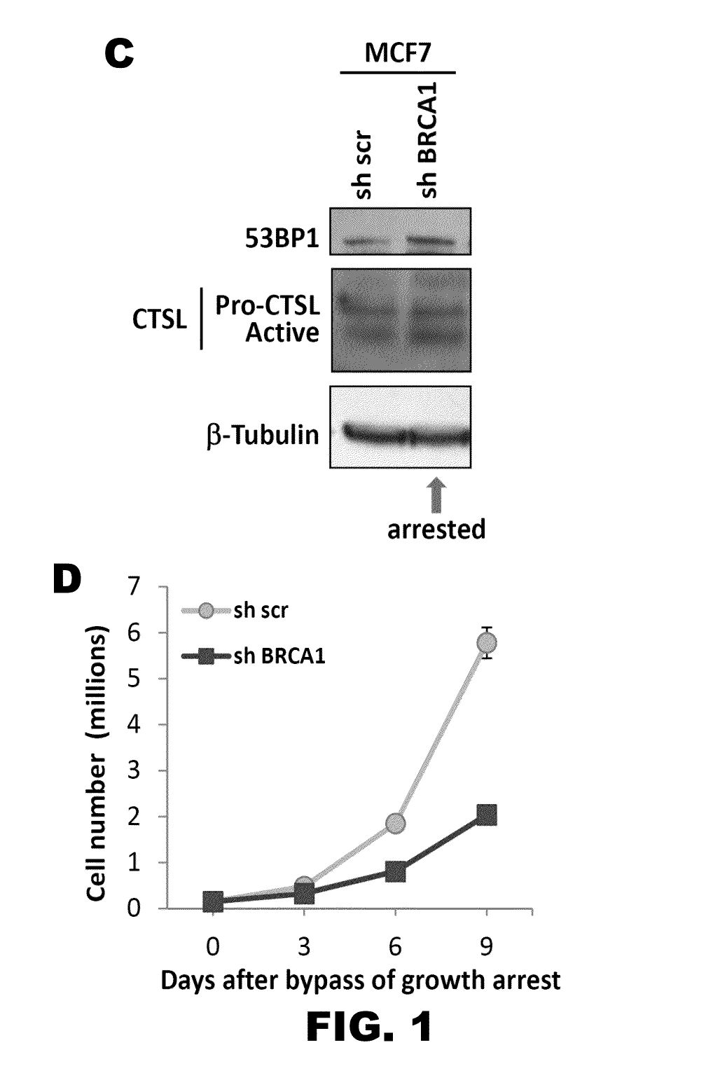

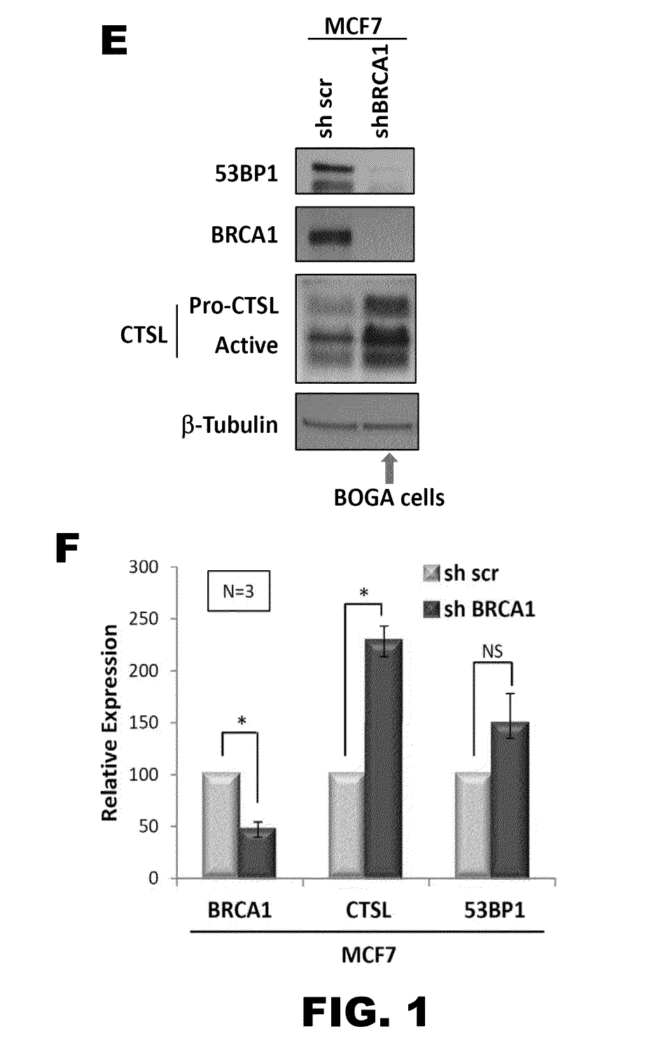

FIG. 1 depicts images of Western blots and graphs showing bypass of growth arrest following BRCA1 loss is associated with upregulation of CTSL and degradation of 53BP1. (A) MCF7 cells were lentivirally transduced with a shRNA specific for BRCA1 or a shRNA control and the levels of BRCA1 assessed by western blot immediately after selection of infected cells with puromycin. (B) Proliferation rate of MCF7 cells proficient or deficient in BRCA1 monitored by counting cell numbers different days after selection of infected cells. Note that depletion of BRCA1 results in growth arrest for up to 14 days. (C) Western blots to monitor the levels of 53BP1 and CTSL (pro-CTSL and active CTSL) in cells arrested following depletion of BRCA1 and control cells. (D) Proliferation rate of BRCA1-deficient cells that overcome growth arrest (here in BOGA cells). Cell numbers were counted for up to 9 days after BOGA cells resume proliferation. (E) Western blots showing the levels of BRCA1, 53BP1 and CTSL in control and BOGA cells. Note the marked upregulation of active CTSL and the low levels of 53BP1. This is a representative experiment out of 10 biological repeats. (F) Graph shows the relative expression of BRCA1, CTSL and 53BP1 in control and BOGA cells as determined by qRT-PCR. The average ±standard deviation of three independent experiments is shown. *p value of statistical significance (*p≦0.05). NS, no statistically significant differences. β-Tubulin was used in all westerns as loading control.

FIG. 2 depicts an image of a Western blot and graphs showing activation of CTSL-mediated degradation of 53BP1 in BRCA1-deficient breast cancer cells. (A) MDA-MB-231 breast cancer cells were lentivirally transduced with a shRNA specific for depletion of BRCA1 or a shRNA scramble (sh scr). Shortly after selection, BRCA1-deficient cells underwent growth arrest. Approximately 10 days later, MDA-MB-231 cells resumed proliferation. Western blots performed in cells that bypass growth arrest (BOGA cells) show upregulation of CTSL and degradation of 53BP1. Thus, loss of BRCA1 activates CTSL-mediated degradation of 53BP1 in breast cancer cells of different types. (Band C) MCF7 control and BOGA cells were lentivirally transduced with a shRNA specific for depletion of CTSL or a shRNA control. Graphs show the relative expression of CTSL (B) and BRCA1 (C) in the different cell lines as determined by qRT-PCR. Note the marked downregulation of CTSL transcripts in control and BOGA cells transduced with shCTSL.

FIG. 3 depicts images of Western blots and graphs showing CTSL is responsible for the degradation of 53BP1 following depletion of BRCA1. (A and B) Control and BOGA cells were lentivirally transduced with a shRNA specific for depletion of CTSL or a shRNA control. After dual selection with G418 (sh scr and shBRCA1) and puromycin (sh luc and shCTSL), the levels of CTSL (A), BRCA1 (A), and 53BP1 (B) were monitored by western blot. Note how depletion of CTSL rescues the levels of 53BP1 in BRCA1-deficient cells. (C) Control or BOGA cells growing in culture were treated with vitamin D (10−7M) or vehicle control (BGS) and the levels of 53BP1 monitored by western blot. The levels of p107, a target of CTSL-mediated degradation, were used as control of CTSL activity. (D) Control or BOGA cells were incubated with the broad cathepsin inhibitor E64 (10 μM) or with vehicle control (H2O), and the levels of 53BP1 and CTSL monitored by western blot. Note how inhibition of cathepsin activity stabilizes 53BP1 protein. Representative experiments out of two biological repeats are shown. β-Tubulin was used in all westerns as loading control.

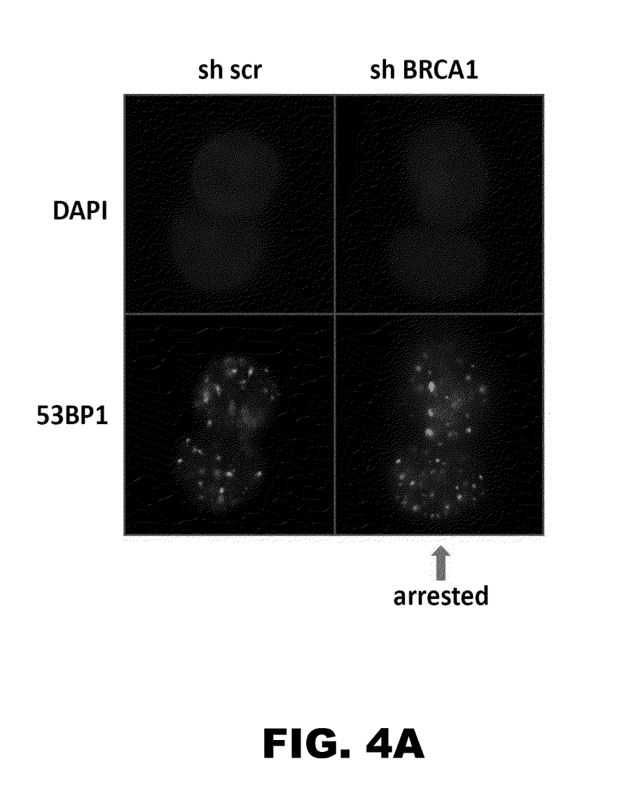

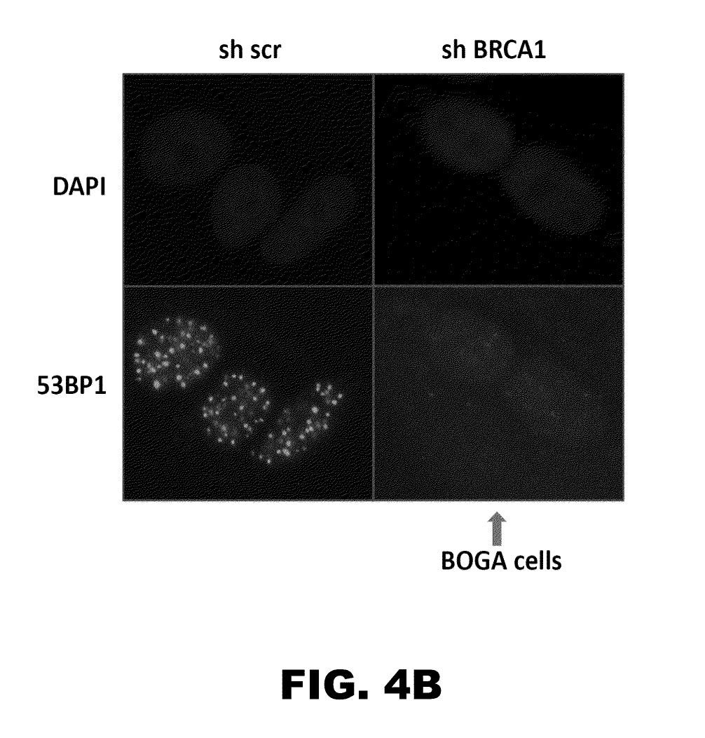

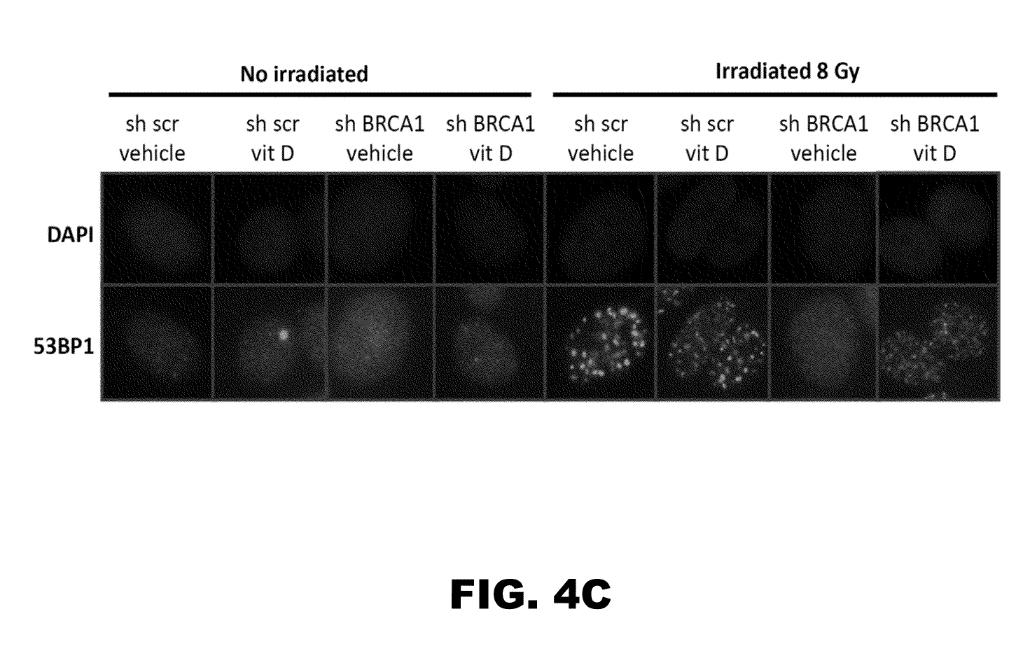

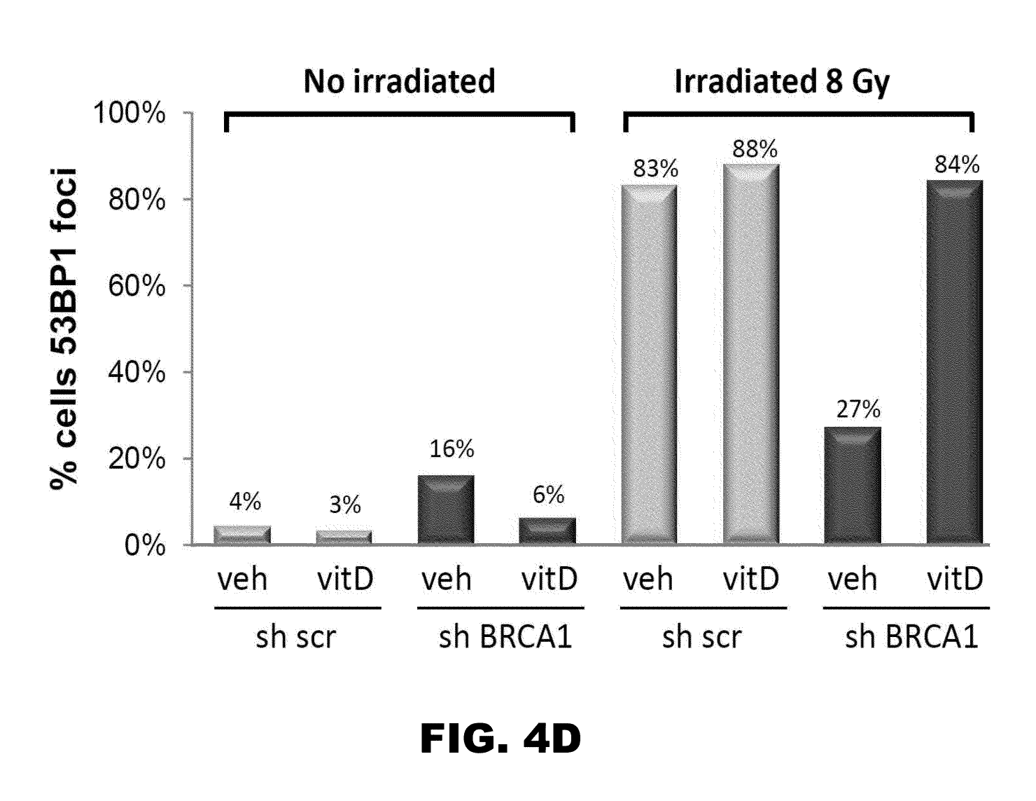

FIG. 4 depicts micrograph images and graphs showing defects in the formation of 53BP1 IRIF in BOGA cells are rescued by vitamin D treatment. (A) MCF7 cells lentivirally transduced with sh scr or sh BRCA1 were irradiated with 8 Gy and subjected to immunofluorescence with 53BP1 antibody following growth arrest of BRCA1-deficient cells. No differences in the ability to form 53BP1 IRIF were observed. DAPI was used for nuclear staining. (B) Immunofluorescence performed in control and BOGA cells. Note how BOGA cells are unable to form 53BP1 IRIF. (C) Immunofluorescence studies performed in control and BOGA cells treated with vitamin D or vehicle starting 24 hours prior to irradiation with 8 Gy. Note how treatment with vitamin D restores the ability of BOGA cells to form 53BP1 IRIF. (D) Graph showing quantitation of the percentage of cells that respond to radiation by forming 53BP1 IRIF in the presence of vitamin D or vehicle control. At least 1000 cells were counted per condition in two independent experiments.

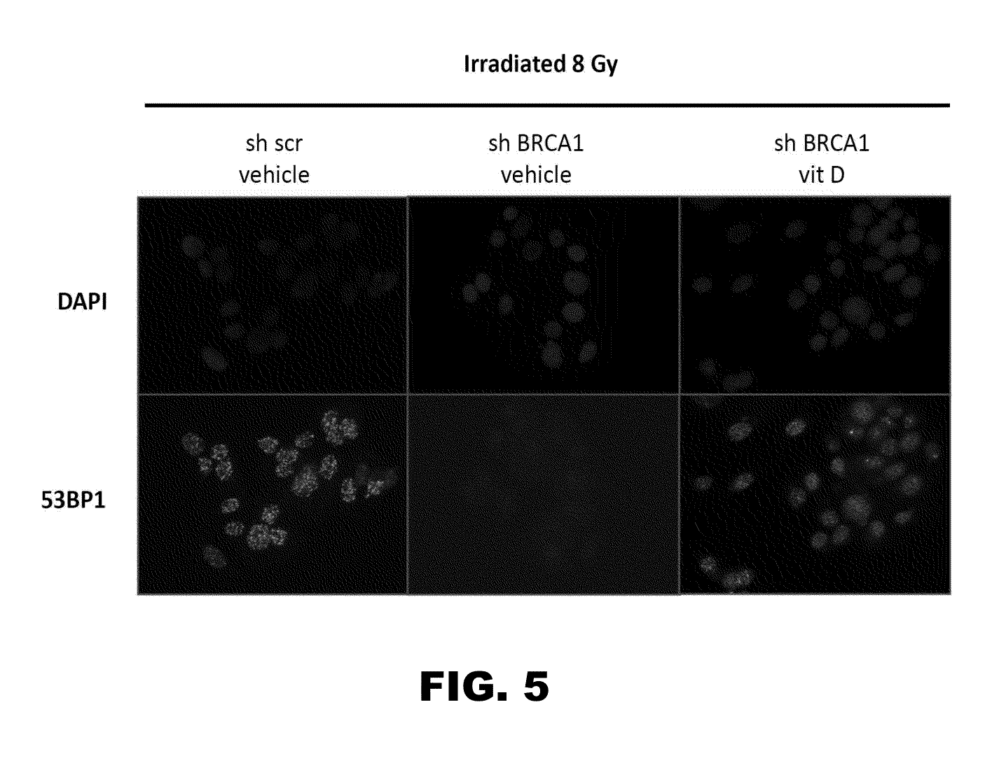

FIG. 5 depicts micrographs showing vitamin D rescues 53BP1 IRIF in BRCA1-deficient cells. Immunofluorescence studies showing the formation of 53BP1 IRIF in control cells (sh scr) and BOGA cells (shBRCA1). Cells were irradiated with 8 Gy and fixed 6 hours post-IR. More than 1000 cells were counted per condition. This is a representative experiment from two biological repeats. The images of large fields show that the effect is not restricted to a few cells, but rather is representative of the whole population of cells. Quantitative data is presented in Table 1.

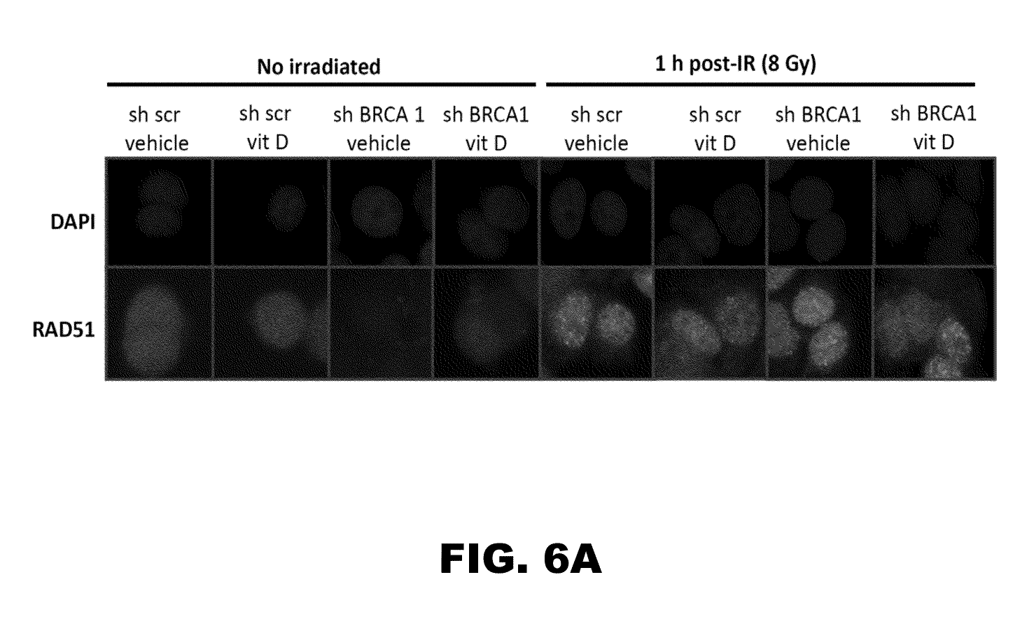

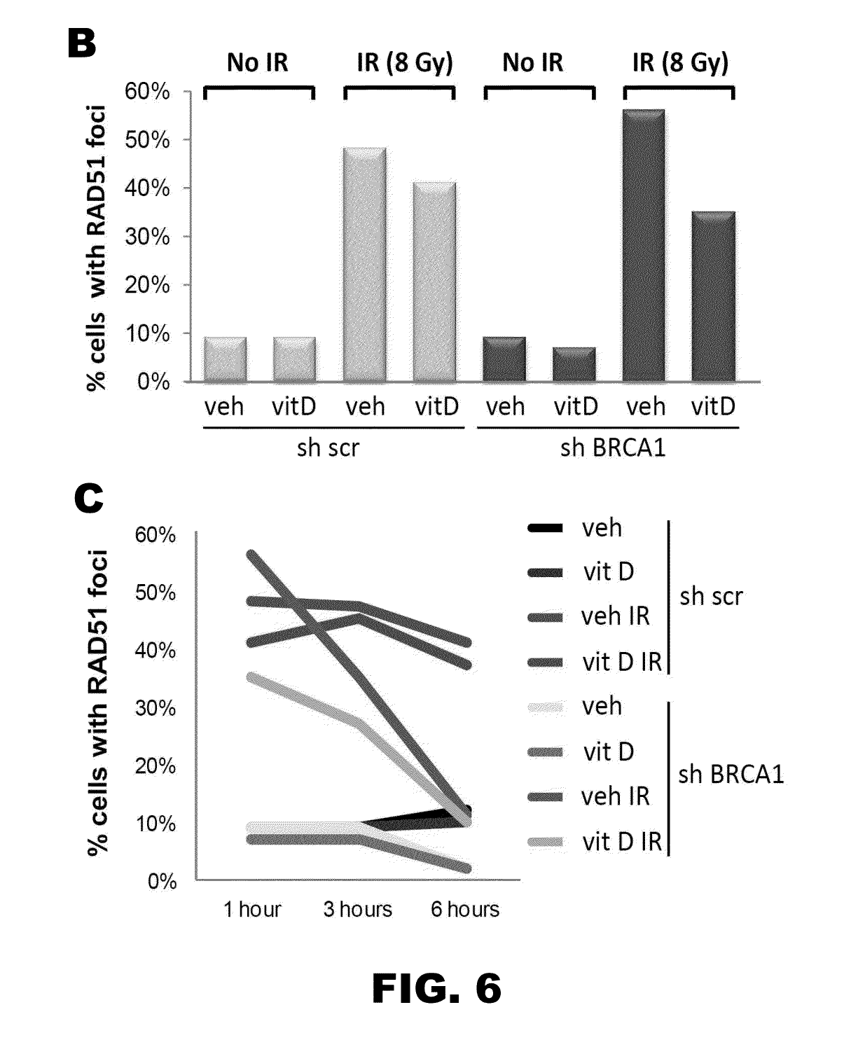

FIG. 6 depicts micrographs and graphs showing regulation of RAD51 IRIF in BOGA cells by vitamin D treatment. (A) Immunofluorescence studies of RAD51 foci formation after 8 Gy of radiation were performed in control and BOGA cells following treatment with vitamin D or vehicle control. Note how BOGA cells are able to form RAD51 foci 1 hour post-IR and how vitamin D partially inhibits RAD51 foci formation. (B) Graph showing quantitation of the percentage of cells that respond to radiation by forming RAD51 IRIF 1 hour post-IR and in the presence of vitamin D or vehicle control. At least 1000 cells were counted per condition in two independent experiments. (C) Quantitation of percentage of cells positive for RAD51 IRIF at more than one timepoint post-IR (1 h, 3 h, and 6 h) and upon treatment with vitamin D or vehicle. Note how BOGA cells exhibit defects in RAD51 IRIF 3 h and 6 h post-IR.

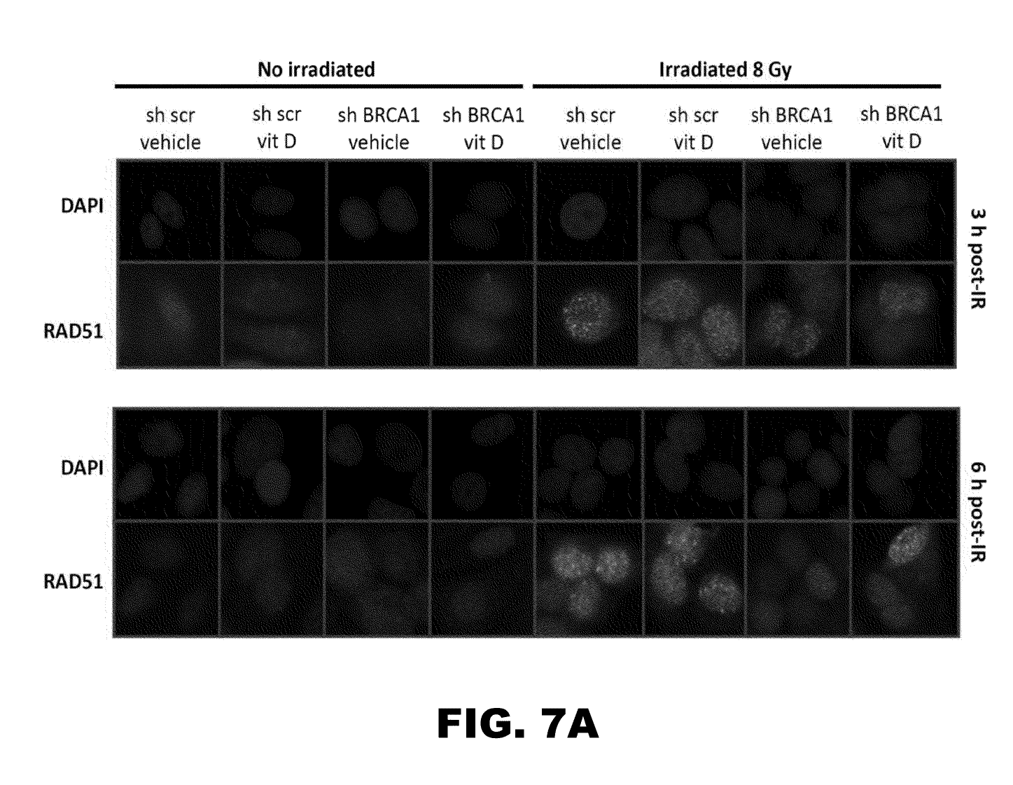

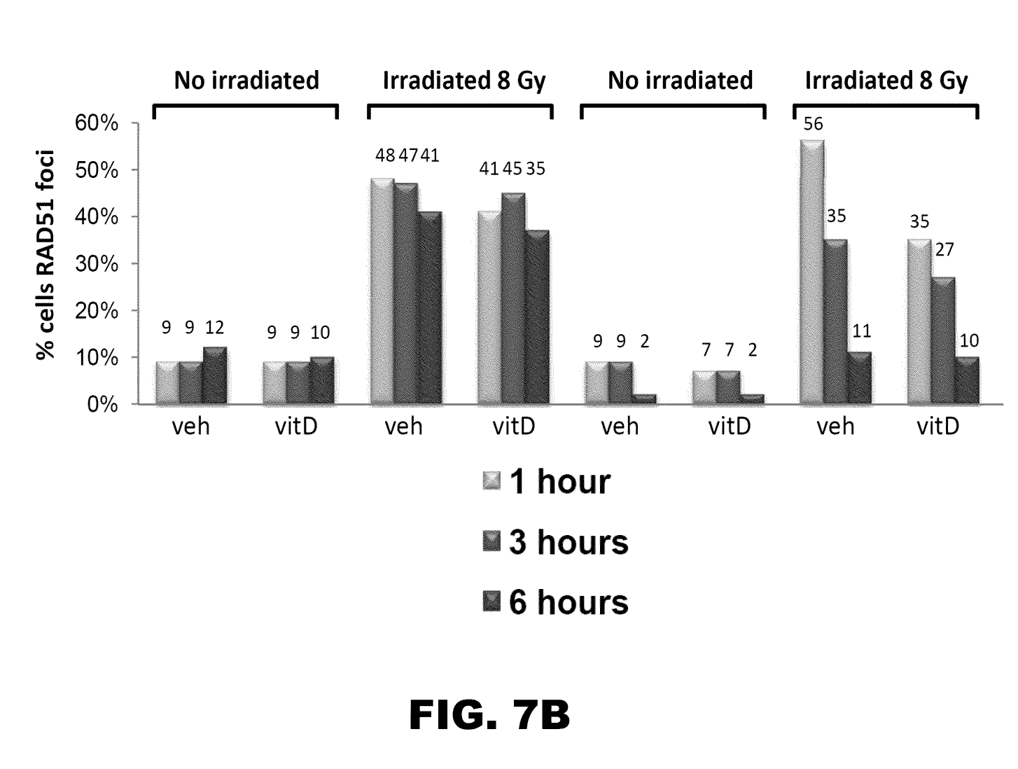

FIG. 7 depicts micrographs and graphs showing the formation of RAD51 IRIF in the context of BRCA1 deficiency. (A) Immunofluorescence images showing the formation of RAD51 IRIF in control and BOGA cells at 3 and 6 hours post-irradiation with 8 Gy. Each of the cell lines was treated with vitamin D or vehicle 24 hours prior to irradiation. Note the decrease in RAD51 foci formation at 3 and 6 hours post-IR. (B) Graph shows quantitation of percentage of control and BOGA cells positive for RAD51 IRIF 1 hour, 3 hours, or 6 hours post-irradiation. Note that while vitamin D impacts on RAD51 foci formation at 1 hour post-IR, it does not exacerbate the defects i at these later time points. At least 1000 cells were counted per condition in two independent experiments.

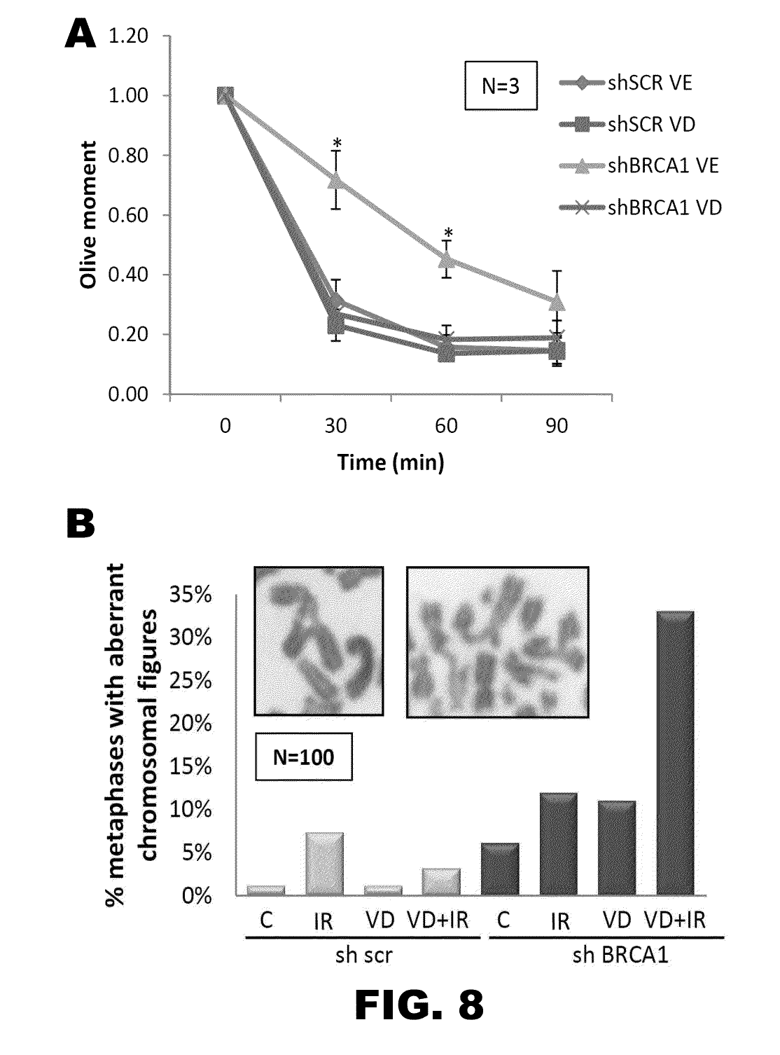

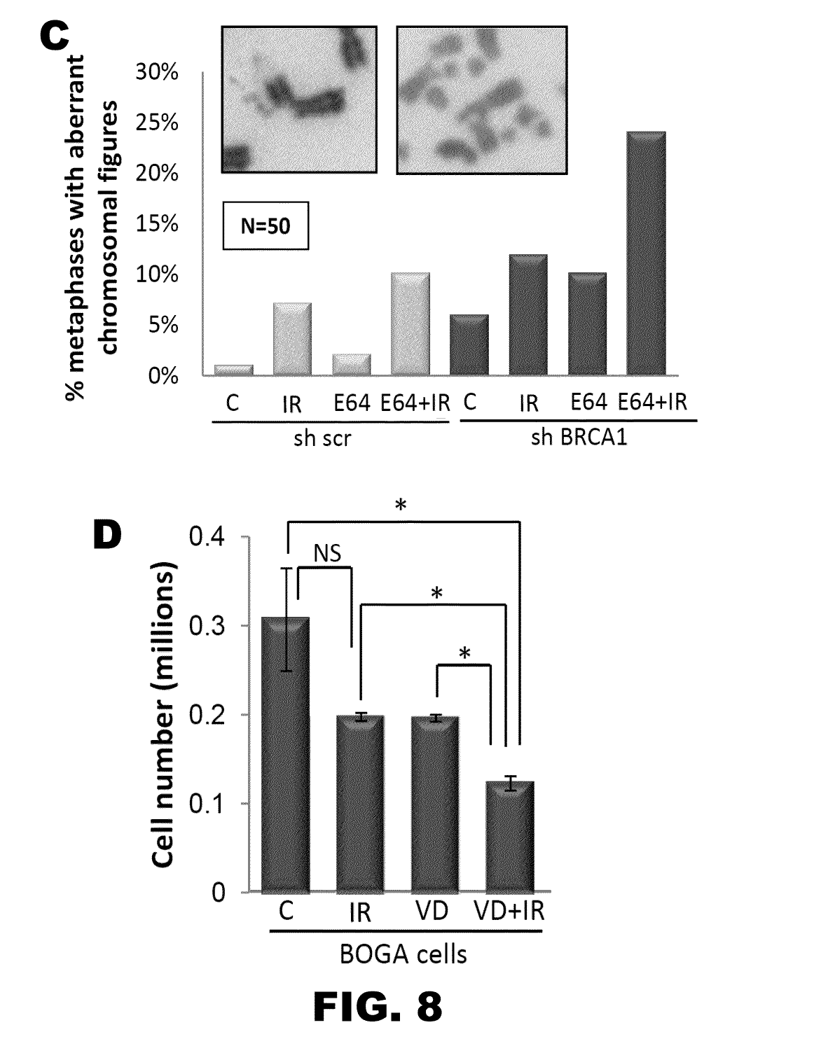

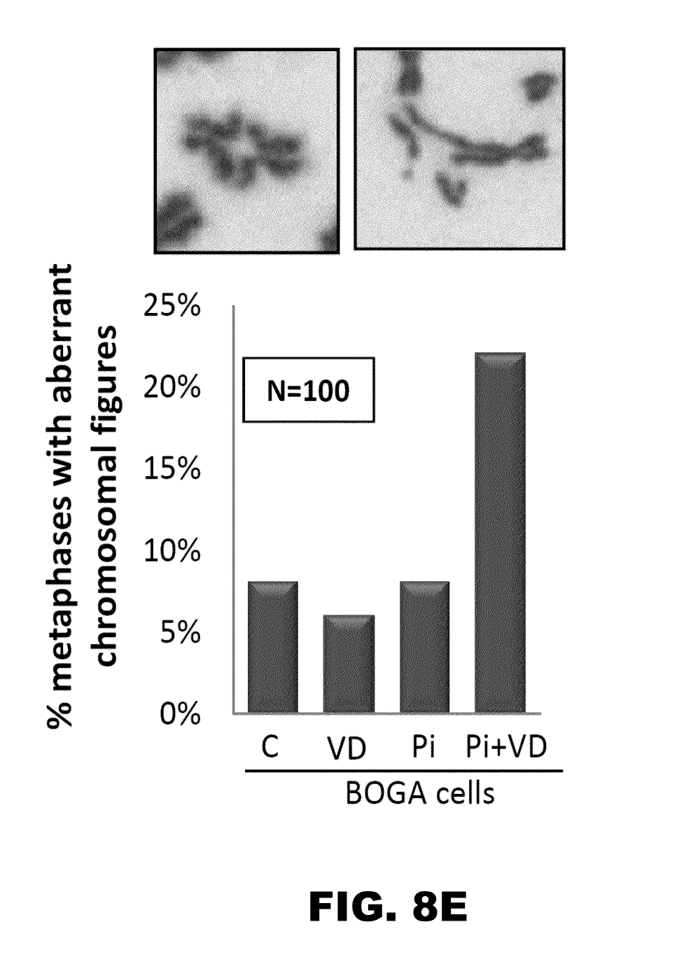

FIG. 8 depicts micrographs and graphs showing the effect of CTSL inhibition on DNA repair and genomic stability in BOGA cells. (A) Neutral comet assays performed after irradiation with 8 Gy show defects in the fast-phase of DNA repair in BOGA cells. Inhibition of CTSL activity by treatment with vitamin D (10−7 M) 24 h preirradiation rescues defects in DNA DSBs repair. Values expressed as mean±SEM. N, number of independent experiments; *p value of statistical significance (*p0.05). (B) Control and BOGA cells were incubated with vehicle (C, control) or vitamin D (VD) for 24 h prior to mock irradiation or irradiation with 2 Gy (IR). Cells collected 24 h post-IR were analyzed for genomic instability by quantitating the percentage of metaphases presenting with chromosomal aberrations. While IR or vitamin D as single agents induce a modest increase in genomic instability in BOGA cells, the combination of vitamin D and IR induced a marked increase in genomic instability. Images show the types of chromosomal aberrations observed in metaphase spreads. N, number of metaphases analyzed. (C) Control and BOGA cells were incubated with the cathepsin inhibitor E64 or vehicle (C, control) for 24 h prior to irradiation with 2 Gy, and the extent of genomic instability assessed as in (B). Note the marked increase in genomic instability upon combined treatment with E64 and IR. (D) Graph shows determination of total numbers of BOGA cells 4 days after treatment with IR, vitamin D, or the combination of both. A total of 150,000 cells were plated initially. Note the decrease in cell number after combined treatment with vitamin D and IR compared to control untreated cells. Values expressed as mean±SEM. *p value of statistical significance (*p≦0.05). NS, no statistically significant differences. (E) BOGA cells were treated with vitamin D (10−7 M) to stabilize 53BP1 24 h prior to treatment with PARPi EB-47 (Pi, 1.2 μg/mL) for an additional 48 h. The percentage of metaphases presenting chromosomal aberrations is shown in the graph. The images show the types of chromosomal aberrations observed. Note the increased genomic instability in cells treated with both PARPi and vitamin D. N, number of metaphases analyzed.

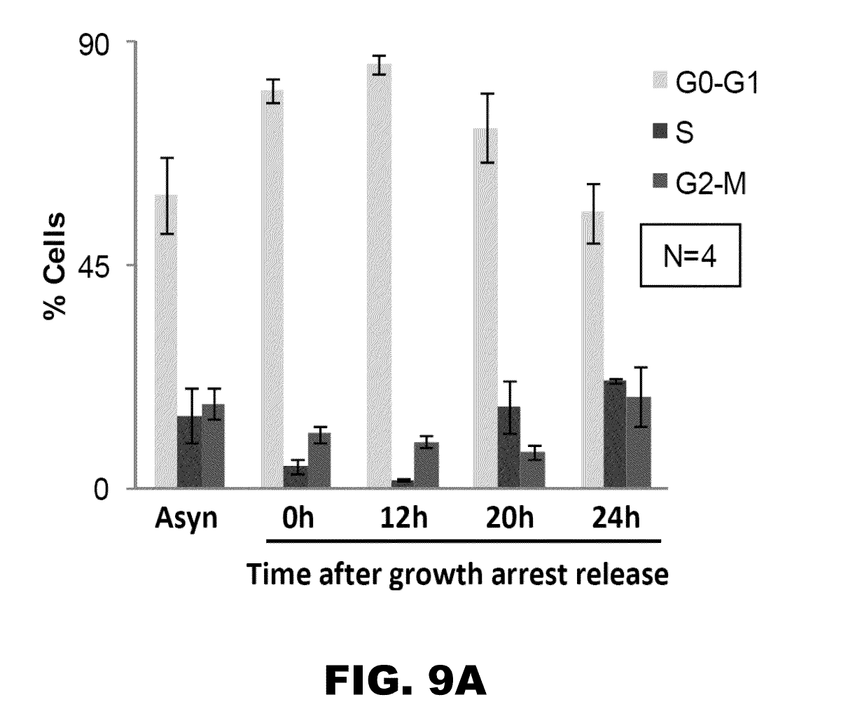

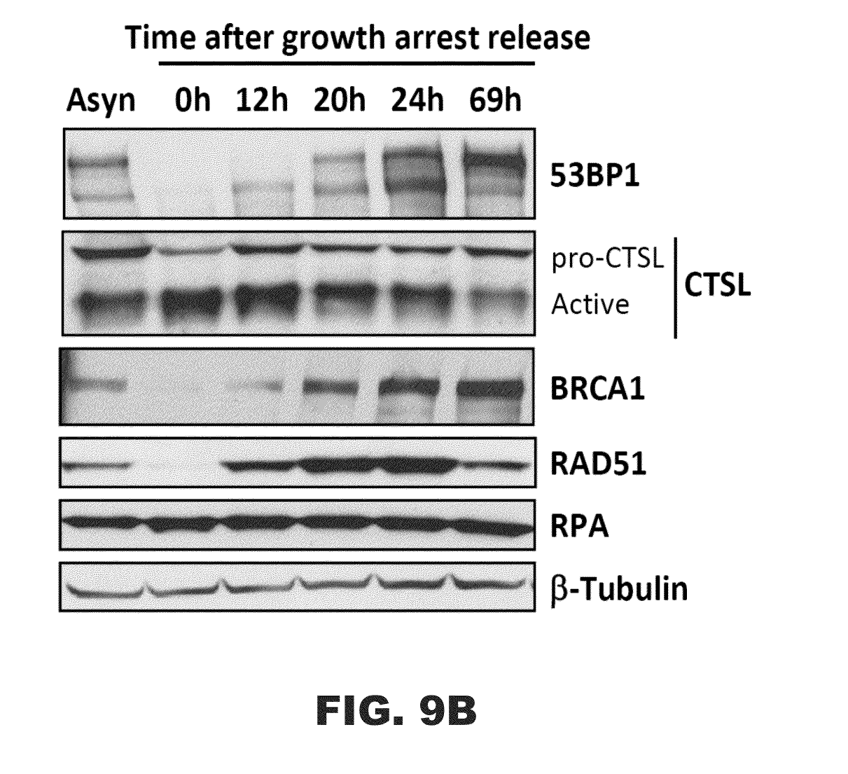

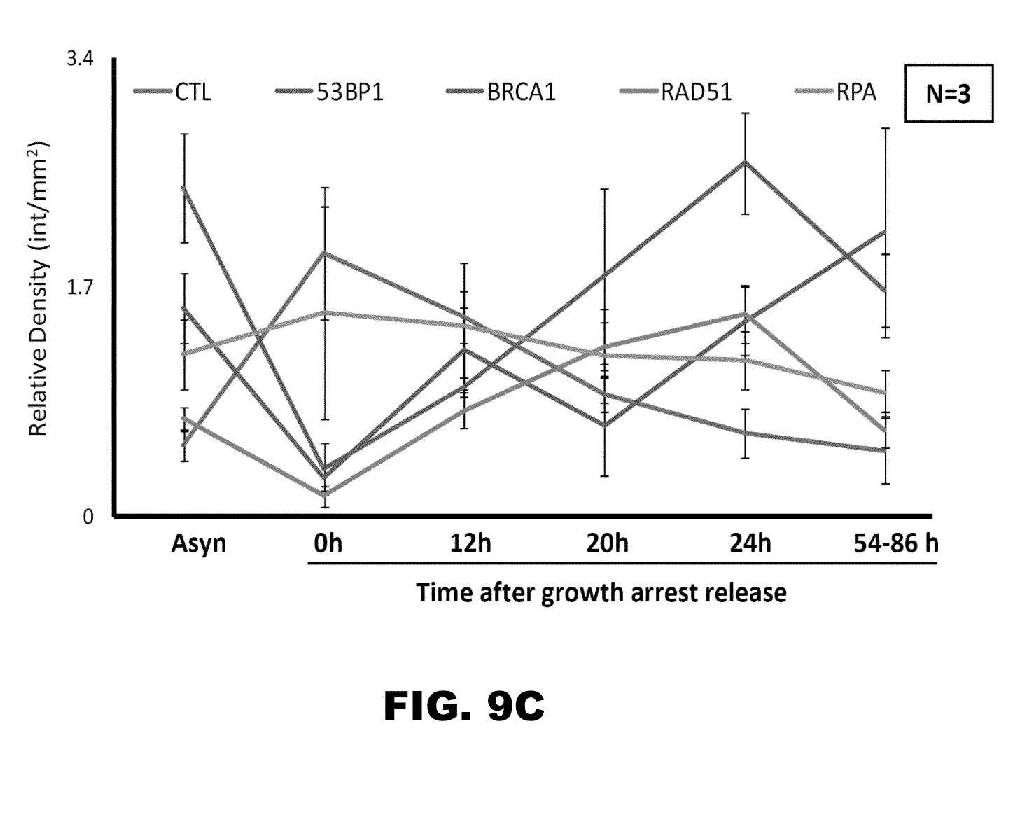

FIG. 9 depicts images of western blots and graphs showing levels of CTSL, BRCA1 and 53BP1 during the cell cycle. (A) Cell cycle profile of human fibroblasts immortalized with telomerase (BJ+hTert) that were collected either while growing asynchronously (Asyn), upon growth arrest by contact inhibition (0 h), or at different times after growth arrest release (12 h, 20 h, or 24 h). Average ±standard deviation of 4 independent experiments. (B) Western blots showing the levels of 53BP1, CTSL, BRCA1, RAD51, RPA, and β-Tubulin at different stages during the cell cycle. Note the inverse correlation between CTSL levels and levels of both 53BP1 and BRCA1. (C) Graph showing the quantitation (average ±standard deviation) of levels of all the proteins in 3 independent experiments. (D) BJ+hTert fibroblasts were lentivirally transduced with a shRNA specific for depletion of CTSL or a shRNA control. After selection, fibroblasts were growth arrested in G0/G1 by contact inhibition. The levels of CTSL, BRCA1, and 53BP1 were monitored by western blot in growth arrested cells. Note how depletion of CTSL increases the levels of both 53BP1 and BRCA1 levels.

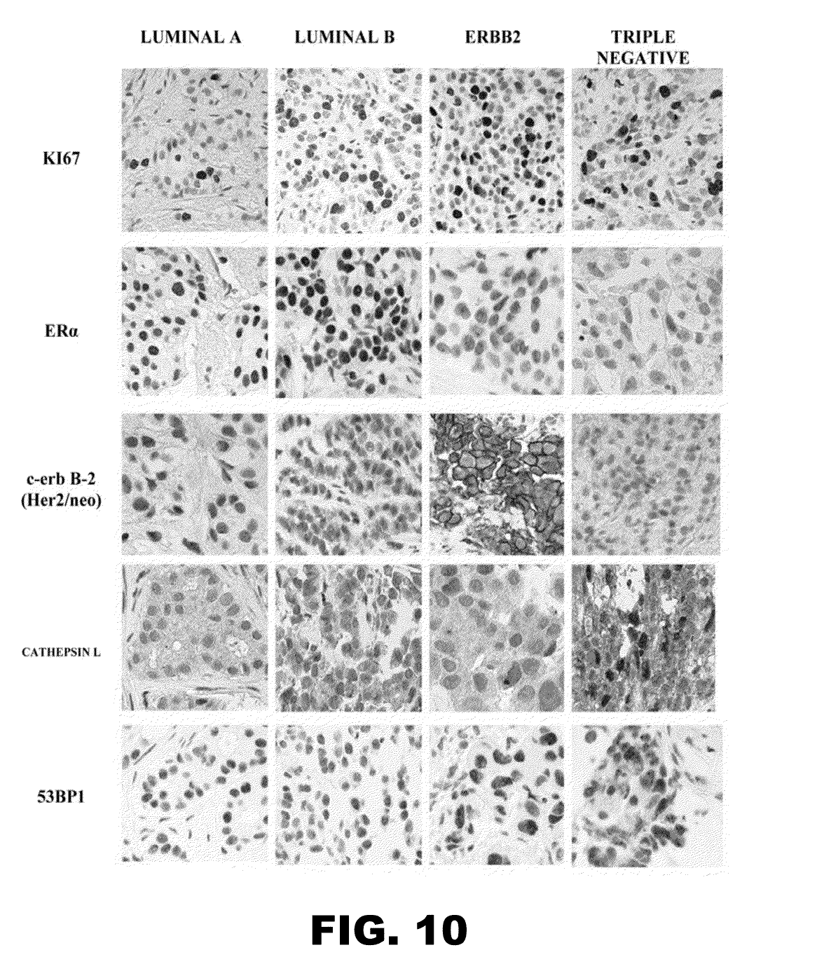

FIG. 10 depicts micrographs showing high levels of nuclear CTSL and low levels of nuclear 53BP1 in a subset of TNBC. Immunohistochemical analysis was performed in breast tumor tissue microarrays from 180 patients which included four molecular subtypes: Luminal A, Luminal B, ERBB2, and Triple-Negative. Representative images of IHC labeling with Ki67, ER, Her2, CTSL and 53BP1 is shown. Note that while cytoplasmic CTSL is observed in all tumor subtypes, nuclear CTSL is markedly upregulated in a subset of TNBC. In addition, TNBC exhibit a marked decrease in 53BP1 labeling.

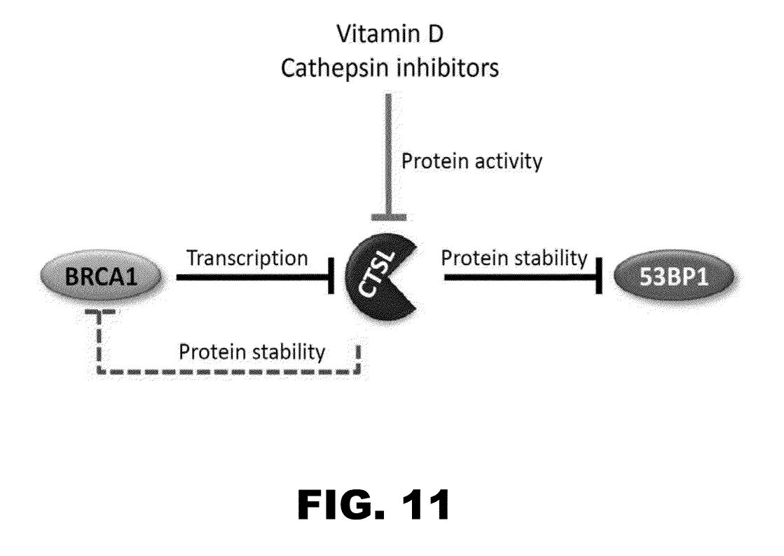

FIG. 11 depicts an illustration showing the functional relationship between BRCA1, CTSL, 53BP1 and Vitamin D.

### DETAILED DESCRIPTION OF THE INVENTION

The present invention provides means for identifying or classifying breast tumors based on the levels of nuclear cathepsin-L (CTSL) and nuclear p53 binding protein (53BP1). The functional relationship between levels of nuclear CTSL and downregulation or degradation of 53BP1 is described in detail below, as is the use of levels of these proteins to predict response to certain types of therapy.

I. Method for Classifying a Breast Tumor Obtained from a Subject

A hallmark of most tumors is genomic instability. Genomic instability due to defective repair of DNA double-strand breaks (DSBs) is one of the underlying causes of breast tumors of the present invention. 53BP1 is a factor in DNA DSB repair and its deficiency is associated with genomic instability and cancer progression. Prior to the present invention, very little information was known as to how the levels of 53BP1 are regulated in normal or tumor cells. The Applicants have demonstrated a heretofor unprecedented role for the cysteine protease Cathepsin L (CTSL) in the degradation of 53BP1 in normal cells. These findings are described in Gonzalez-Suarez et al. EMBO Journal (2011) 30: 33383-3396 and Redwood et al. Cell Cycle (2011) 10(10): 3652-3657, each hereby incorporated by reference in its entirety. As detailed further in the Examples, the Applicants have also discovered that CTSL plays a role in 53BP1 downregulation in some, but not all, BRCA1-deficient/triple negative breast tumors, and that up-regulation of CTSL-mediated degradation of 53BP1 is a new biomarker for a subset of breast tumors that are less likely to be responsive to DNA-damaging agents.

One aspect of the invention provides a method for classifying a breast tumor in a subject. Typically, the method comprises obtaining a sample of a breast tumor from a subject, processing the sample in vitro to determine the levels of nuclear CTSL and nuclear 53BP1 in at least one tumor cell comprising the sample, and classifying the tumor as having (i) high nuclear CTSL and low nuclear 53BP1, (ii) high nuclear CTSL and high nuclear 53BP1, or (iii) low nuclear CTSL and low nuclear 53BP1.

Another aspect of the invention provides a method for classifying a breast tumor in a subject as having or not having CTSL-mediated downregulation of 53BP1. Typically, the method comprises obtaining a sample of a breast tumor from a subject, processing the sample in vitro to determine the levels of nuclear CTSL and nuclear 53BP1 in at least one tumor cell comprising the sample, and classifying the tumor as having CTSL-mediated downregulation of 53BP1 if the sample has high nuclear CTSL and low nuclear 53BP1 or as not having CTSL-mediated downregulation of 53BP1 if the sample has either (i) high nuclear CTSL and high nuclear 53BP1, or (ii) low nuclear CTSL and low nuclear 53BP1.

Another aspect of the invention provides a method for classifying a breast tumor in a subject as having or not having CTSL-mediated degradation of 53BP1. Typically, the method comprises obtaining a sample of a breast tumor from a subject, processing the sample in vitro to determine the levels of nuclear CTSL and nuclear 53BP1 in at least one tumor cell comprising the sample, and classifying the tumor as having CTSL-mediated degradation of 53BP1 if the sample has high nuclear CTSL and low nuclear 53BP1 or as not having CTSL-mediated degradation of 53BP1 if the sample has either (i) high nuclear CTSL and high nuclear 53BP1, or (ii) low nuclear CTSL and low nuclear 53BP1.

Methods of processing the sample in vitro to determine the levels of nuclear CTSL and nuclear 53BP1 are described in detail below. In some embodiments, the method is selected from the group consisting of immunohistochemistry, flow cytometry, array and ELISA. In other embodiments, the method is mass spectrometry. Suitable breast tumors, samples, subjects and levels are also described below.

### A. Breast Tumors of the Present Invention

One aspect of the invention provides for a group of breast tumors consisting of basal-like tumors, triple negative breast tumors, BRCA1-deficient tumors, and combinations thereof. This group of breast tumors is associated with high grade, poor prognosis and younger patient age. Breast tumors are heterogeneous and may be classified by any system known in the art. Non-limiting examples may include gene expression profiling, BRCA1 and/or BRCA2 mutational status, and human epidermal growth factor receptor-2 (HER2), estrogen receptor (ER) and progesterone receptor (PR) status.

Gene expression profiling has been shown to classify breast tumors into five major biologically distinct subtypes: luminal A, luminal B, human epidermal growth factor receptor-2 (HER2) overexpressing, basal-like, and normal like. For more detail on this classification system, see Perou et al. 2000 Nature 406:747-52, Sorlie et al. 2001 PNAS 98:10869-74, and Sorlie et al. 2003 PNAS 100:8418-23. In some embodiments of the present invention, the breast tumor is a basal-like tumor. Basal-like tumors lack estrogen receptor (ER) and HER2 and express genes characteristic of basal epithelial cells. A breast tumor may be identified or classified as a basal-like tumor based on its gene expression profile. Genes and functional groups characteristically overexpressed or downregulated in basal-like tumors are known in the art. For example, see the review on basal-like tumors published by Rakha et al. in the Journal of Clinical Oncology (2008; 26(15): 2568-2581), hereby incorporated by reference in its entirety.

Breast tumors may also be classified by the presence or absence of mutations in the BRCA1 and/or BRCA2 genes. A skilled artisan may use one or more of the methods known in the art to test for BRCA1 and BRCA2 mutations; see for example Malone et al. 2006 Cancer Research 66(16):8297-8308. Most of these methods look for changes in BRCA1 and BRCA2 DNA. At least one method looks for changes in the proteins produced by these genes. Frequently, a combination of methods is used. In some embodiments of the present invention, the breast tumor is BRCA1-deficient. The terms “BRCA1-deficient cell” and “BRCA1-deficient tumor” refer to a cell or tumor, respectively, comprising one or more BRCA1 mutations that results in the lack of BRCA1. The terms “BRCA2-deficient cell” and “BRCA2-deficient tumor” refer to a cell or tumor, respectively, comprising one or more BRCA2 mutations that results in the lack of BRCA2. Cells and tumors that lack both BRCA1 and BRCA1 function are termed “BRCA1/2-deficient”.

Breast tumors may also be classified based on the presence or absence of the three most common types of receptors known to fuel breast tumor growth. A triple negative breast tumor is negative for HER2, ER, and progesterone receptors (PR). Methods of identifying a triple negative breast tumor are well known in the art. In some embodiments of the present invention, the breast tumor is a triple negative breast tumor.

Breast tumors may be classified in more than one way, meaning the same breast tumor may be classified using two or more different classification systems resulting in two or more different classifications. For example, a breast tumor may be classified as a basal-like tumor and a triple negative tumor. In a different example, a breast tumor may be classified only as a basal-like tumor and not a triple negative tumor or a BRCA1-deficient tumor. Breast tumors of the present invention may have at least one, at least two, or at least three different classifications.

### B. Subset of Breast Tumors

Another aspect of the invention provides for a subset of breast tumors within a group of breast tumors. In some embodiments, a subset comprises breast tumors with high levels of nuclear CTSL and low levels of nuclear 53BP1. High levels of nuclear CTSL and low levels of nuclear 53BP1 in a tumor indicate that CTSL-mediated downregulation of 53BP1 is occurring in that tumor. High levels of nuclear CTSL and low levels of nuclear 53BP1 in a tumor also indicate that CTSL-mediated degradation of 53BP1 is occurring in that tumor. When CTSL-mediated downregulation or degradation of 53BP1 is occurring in a breast tumor of the invention, DNA-damaging agents may be less likely to inhibit tumor growth. This is because CTSL-mediated degradation of 53BP1 in the tumor cells comprising the breast tumor (i) rescues DNA repair defects, (ii) rescues proliferation, (iii) rescues viability, (iv) decreases genomic instability, or (v) a combination thereof.

In other embodiments, a subset comprises breast tumors with low levels of nuclear CTSL and low levels of nuclear 53BP1. Low levels of nuclear CTSL and low levels of nuclear 53BP1 in a tumor indicate that CTSL-mediated downregulation of 53BP1 is not occurring in that tumor. Low levels of nuclear CTSL and low levels of nuclear 53BP1 in a tumor also indicate that CTSL-mediated degradation of 53BP1 is not occurring in that tumor. When CTSL-mediated downregulation or degradation of 53BP1 is not occurring in a breast tumor of the invention, DNA-damaging agents may be likely to inhibit tumor growth. This is because in the absence of CTSL-mediated degradation of 53BP1 in the tumor cells comprising the breast tumor, 53BP1 protein levels are stabilized, which (i) increases DNA repair defects, (ii) slows or halts proliferation, (iii) decreases viability, (iv) increases genomic instability, or (v) a combination thereof.

In still other embodiments, a subset comprises breast tumors with high levels of nuclear CTSL and high levels of nuclear 53BP1. High levels of nuclear CTSL and high levels of nuclear 53BP1 in a tumor indicate that CTSL-mediated downregulation of 53BP1 is not occurring in that tumor. High levels of nuclear CTSL and high levels of nuclear 53BP1 in a tumor also indicate that CTSL-mediated degradation of 53BP1 is not occurring in that tumor. Suitable breast tumors are described above. When CTSL-mediated downregulation or degradation of 53BP1 is not occurring in a breast tumor of the invention, DNA-damaging agents may be likely to inhibit tumor growth. This is because in the absence of CTSL-mediated degradation of 53BP1 in the tumor cells comprising the breast tumor, 53BP1 protein levels are stabilized, which (i) increases DNA repair defects, (ii) slows or halts proliferation, (iii) decreases viability, (iv) increases genomic instability, or (v) a combination thereof.

As used herein, the term “levels” refers to a measurement of the amount of protein expression. Thus, the phrases “levels of nuclear CTSL” and “levels of nuclear 53BP1” refer to a measurement of the amount of CTSL and 53BP1 protein expression in the nucleus of tumor cells comprising the tumor. In some embodiments, the measurement is qualitative. In other embodiments, the measurement is semi-quantitative. In still other embodiments, the measurement is a quantitative. Methods for determining an amount of protein expression typically comprise obtaining a sample of the tumor and processing the sample in vitro to determine the amount of protein expression. This aspect of the invention is described in further detail below.

Subsets of breast tumors may be identified based on levels of nuclear CTSL and 53BP1. Briefly, after measuring the amount of nuclear CTSL and 53BP1 protein expression, the statistical significance of the measured differences may be calculated and/or the statistical significance of the relationship between nuclear CTSL and 53BP1 levels may be calculated for all breast tumor types. Through these statistical analyses, a cut-off level (or value) can be identified which discriminates high levels of nuclear CTSL from low levels of nuclear CTSL, and high levels of nuclear 53BP1 from low levels of nuclear 53BP1. Example 6 describes this process in more detail. In some embodiments, the high and low levels of nuclear CTSL and nuclear 53BP1 may be determined from the average level of nuclear CTSL and nuclear 53BP1 protein expression in a population of breast tumors examined, wherein a high level is above the average and a low level is below the average. In other embodiments, the cut-off value may be a median value for each protein in a population of breast tumors examined. A skilled artisan will appreciate that the level of nuclear CTSL and 53BP1 reported (i.e. the absolute value) will be dependent on the method used to measure the amount of protein expression. For example, if the amount of nuclear CTSL and 53BP1 is measured by immunohistochemistry, one method to report protein expression is a semiquantitative measurement (or score) that takes into consideration the percentage and intensity of staining, and also the percentage of positive cells. As a further example, a histological score (Hscore) ranging from 0 (no immune reaction) to 300 (maximal immunoreactivity) may be obtained with the formula: Hscore=1(% light staining)+2(% moderate staining)+3(% strong staining).

In some exemplary embodiments, the cut-off for nuclear CTSL is an Hscore value no greater than about 50, wherein a value above the cut-off indicates high nuclear CTSL and a value below the cut-off indicates low nuclear CTSL. In other embodiments the cut-off for nuclear CTSL is an Hscore value of about 25 to about 50. In still other embodiments the cut-off for nuclear CTSL is an Hscore value of about 0 to about 25. In still other embodiments the cut-off for nuclear CTSL is an Hscore value of about 0 to about 50. For example, the Hscore value may be about 0, about 5, about 10, about 15, about 20, about 25, about 30, about 35, about 40, about 45, or about 50. The Hscore value may also be 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

In other exemplary embodiments, the cut-off for nuclear 53BP1 is an Hscore value no less than about 115, wherein a value above the cut-off indicates high nuclear 53BP1 and a value below the cut-off indicates low nuclear 53BP1. In some embodiments the cut-off for nuclear 53BP1 is an Hscore value of about 115 to about 140. In other embodiments the cut-off for nuclear CTSL is an Hscore value of about 140 to about 165. In still other embodiments the cut-off for nuclear CTSL is an Hscore value of about 115 to about 165. For example, the Hscore value may be about 115, about 120, about 125, about 130, about 135, about 140, about 145, about 150, about 155, about 160, or about 165. The Hscore value may also be 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, or 165.

From the foregoing description, one skilled in the art can easily ascertain comparable cut-off values using other methods suitable for determining an amount of protein expression known in the art.

### C. Methods for Determining an Amount of Protein Expression

Methods for determining an amount of protein expression in tumor cells comprising a tumor are well known in the art, and all suitable methods for determining an amount of protein expression known to one of skill in the art are contemplated within the scope of the invention. Generally, the method comprises obtaining a sample of the tumor and processing the sample in vitro to determine the amount of protein expression.

Suitable samples include, but are not limited to, biological samples and in vitro cell lines. As used herein, “biological sample” refers to a sample derived from a subject. Typically, the subject has a breast tumor, an abnormal lump in a breast, or a metastatic tumor of breast origin. Suitable subjects may include a human, a livestock animal, a companion animal, a laboratory animal, or a zoological animal. In a preferred embodiment, a subject is human.

Numerous types of biological samples are known in the art. Suitable biological sample may include, but are not limited to, tissue samples or bodily fluids. In some embodiments, the biological sample is a tissue sample such as a tissue biopsy. The tissue biopsy may be a biopsy of a breast tumor, an abnormal lump in the breast, or a metastatic tumor of breast origin. The biopsied tissue may be fixed, embedded in paraffin or plastic, and sectioned, or the biopsied tissue may be frozen and cryosectioned. Alternatively, the biopsied tissue may be processed into individual cells or an explant, or processed into a homogenate, a cell extract, a membranous fraction, or a protein extract. The sample may also be primary and/or transformed cell cultures derived from tissue from the subject. In other embodiments, the sample may be a bodily fluid. Non-limiting examples of bodily fluids include blood, serum, plasma, saliva, sputum, lymph, nipple aspirate and ascites. The fluid may be used “as is”, the cellular components may be isolated from the fluid, or a protein faction may be isolated from the fluid using standard techniques.

As will be appreciated by a skilled artisan, the method of collecting a biological sample can and will vary depending upon the nature of the biological sample and the type of analysis to be performed. Any of a variety of methods generally known in the art may be utilized to collect a biological sample. Generally speaking, the method preferably maintains the integrity of the sample such that protein expression can be accurately measured according to the invention.

Once the sample is obtained, it is processed in vitro to determine the amount of protein expression. Non-limiting examples of suitable methods to determine an amount of protein expression may include epitope binding agent-based methods and mass spectrometry based methods.

In some embodiments, the method to determine an amount of protein expression is mass spectrometry. By exploiting the intrinsic properties of mass and charge, mass spectrometry (MS) can resolve and confidently identify a wide variety of complex compounds, including proteins. Traditional quantitative MS has used electrospray ionization (ESI) followed by tandem MS (MS/MS) (Chen et al., 2001; Zhong et al., 2001; Wu et al., 2000) while newer quantitative methods are being developed using matrix assisted laser desorption/ionization (MALDI) followed by time of flight (TOF) MS (Bucknall et al., 2002; Mirgorodskaya et al., 2000; Gobom et al., 2000). In accordance with the present invention, one can use mass spectrometry to look for the levels of 53BP1 and CTSL particularly.

In some embodiments, the method to determine an amount of protein expression is an epitope binding agent-based method. As used herein, the term “epitope binding agent” refers to an antibody, an aptamer, a nucleic acid, an oligonucleic acid, an amino acid, a peptide, a polypeptide, a protein, a lipid, a metabolite, a small molecule, or a fragment thereof that recognizes and is capable of binding to 53BP1, CSTL, or a portion thereof. For example, CTSL is synthesized as an inactive propeptide that undergoes autoproteolytic processing to release active enzyme. In some embodiments, an epitope binding agent recognizes and is capable of binding to the inactive propeptide of CTSL. In other embodiments, an epitope binding agent recognizes and is capable of binding to the active form of CTSL. Nucleic acids may include RNA, DNA, and naturally occurring or synthetically created derivative.

As used herein, the term “antibody” generally means a polypeptide or protein that recognizes and can bind to an epitope of an antigen. An antibody, as used herein, may be a complete antibody as understood in the art, i.e., consisting of two heavy chains and two light chains, or may be any antibody-like molecule that has an antigen binding region, and includes, but is not limited to, antibody fragments such as Fab′, Fab, F(ab′)2, single domain antibodies, Fv, and single chain Fv. The term antibody also refers to a polyclonal antibody, a monoclonal antibody, a chimeric antibody and a humanized antibody. The techniques for preparing and using various antibody-based constructs and fragments are well known in the art. Means for preparing and characterizing antibodies are also well known in the art (See, e.g. Antibodies: A Laboratory Manual, Cold Spring Harbor Laboratory, 1988; herein incorporated by reference in its entirety).

As used herein, the term “aptamer” refers to a polynucleotide, generally a RNA or DNA that has a useful biological activity in terms of biochemical activity, molecular recognition or binding attributes. Usually, an aptamer has a molecular activity such as binging to a target molecule at a specific epitope (region). It is generally accepted that an aptamer, which is specific in it binding to a polypeptide, may be synthesized and/or identified by in vitro evolution methods. Means for preparing and characterizing aptamers, including by in vitro evolution methods, are well know in the art (See, e.g. U.S. Pat. No. 7,939,313; herein incorporated by reference in its entirety).

In general, an epitope binding agent-based method of determining an amount of protein expression comprises contacting a sample comprising a polypeptide with an epitope binding agent specific for the polypeptide under conditions effective to allow for formation of a complex between the epitope binding agent and the polypeptide. Epitope binding agent-based methods may occur in solution, or the epitope binding agent or sample may be immobilized on a solid surface. Non-limiting examples of suitable surfaces include microtitre plates, test tubes, beads, resins, and other polymers.

An epitope binding agent may be attached to the substrate in a wide variety of ways, as will be appreciated by those in the art. The epitope binding agent may either be synthesized first, with subsequent attachment to the substrate, or may be directly synthesized on the substrate. The substrate and the epitope binding agent may be derivatized with chemical functional groups for subsequent attachment of the two. For example, the substrate may be derivatized with a chemical functional group including, but not limited to, amino groups, carboxyl groups, oxo groups or thiol groups. Using these functional groups, the epitope binding agent may be attached directly using the functional groups or indirectly using linkers.

The epitope binding agent may also be attached to the substrate non-covalently. For example, a biotinylated epitope binding agent may be prepared, which may bind to surfaces covalently coated with streptavidin, resulting in attachment. Alternatively, an epitope binding agent may be synthesized on the surface using techniques such as photopolymerization and photolithography. Additional methods of attaching epitope binding agents to solid surfaces and methods of synthesizing biomolecules on substrates are well known in the art, i.e. VLSIPS technology from Affymetrix (e.g., see U.S. Pat. No. 6,566,495, and Rockett and Dix, Xenobiotica 30(2):155-177, both of which are hereby incorporated by reference in their entirety).

Contacting the sample with an epitope binding agent under effective conditions for a period of time sufficient to allow formation of a complex generally involves adding the epitope binding agent composition to the sample and incubating the mixture for a period of time long enough for the epitope binding agent to bind to any antigen present. After this time, the complex will be washed and the complex may be detected by any method well known in the art. Methods of detecting the epitope binding agent-polypeptide complex are generally based on the detection of a label or marker. The term “label”, as used herein, refers to any substance attached to an epitope binding agent, or other substrate material, in which the substance is detectable by a detection method. Non-limiting examples of suitable labels include luminescent molecules, chemiluminescent molecules, fluorochromes, fluorescent quenching agents, colored molecules, radioisotopes, scintillants, biotin, avidin, stretpavidin, protein A, protein G, antibodies or fragments thereof, polyhistidine, Ni²⁺, Flag tags, myc tags, heavy metals, and enzymes (including alkaline phosphatase, peroxidase, and luciferase). Methods of detecting an epitope binding agent-polypeptide complex based on the detection of a label or marker are well known in the art.

In some embodiments, the epitope binding agent-based method is an ELISA. In other embodiments, the epitope binding agent-based method is a radioimmunoassay. In still other embodiments, the epitope binding agent-based method is an immunoblot or Western blot. In yet other embodiments, the epitope binding agent-based method is flow cytometry.

In different embodiments, the epitope binding agent-based method is immunohistochemistry (IHC). IHC uses epitope binding agents to detect and quantify antigens in intact tissue samples. The tissue samples may be fresh-frozen and/or formalin-fixed, paraffin-embedded (or plastic-embedded) tissue blocks prepared for study by IHC. Methods of preparing tissue block for study by IHC, as well as methods of performing IHC are well known in the art. Further details may also be found in the Examples.

In alternative embodiments, the epitope binding agent-based method is an array. An array comprises at least one address, wherein at least one address of the array has disposed thereon an epitope binding agent. Several substrates suitable for the construction of arrays are known in the art, and one skilled in the art will appreciate that other substrates may become available as the art progresses. Suitable substrates are also described above. In one embodiment, the epitope binding agent attached to the substrate is located at a spatially defined address of the array. Arrays may comprise from about 1 to about several hundred thousand addresses. In one embodiment, the array may be comprised of less than 10,000 addresses. In another alternative embodiment, the array may be comprised of at least 10,000 addresses. In yet another alternative embodiment, the array may be comprised of less than 5,000 addresses. In still another alternative embodiment the array may be comprised of at least 5,000 addresses. In a further embodiment, the array may be comprised of less than 500 addresses. In yet a further embodiment, the array may be comprised of at least 500 addresses.

### II. Method for Increasing the Sensitivity of a Tumor Cell to DNA-Damaging Agent

Another aspect of the invention provides a method for increasing the sensitivity of a tumor cell to a DNA-damaging agent. Typically, the method comprises contacting the tumor cell with an effective amount of a CTSL inhibitor and a DNA-damaging agent. The tumor cell may be an immortalized cell, a primary cell isolate, or may be one of several tumor cells that comprise a breast tumor. In preferred embodiment, the tumor cell is in a breast tumor in a subject.

Suitable CTSL inhibitors are known in the art. See for example, U.S. Pat. No. 6,004,933, U.S. Pat. No. 8,232,240, U.S. Pat. No. 5,374,623, US 201110207726 and Sudhan et al. Molecular Cancer Therapeutics (2011) 10(11): Supplement 1. In some embodiments, the CTSL inhibitor may be specific for CTSL (i.e. it does not appreciably inhibit other cysteine proteases). In other embodiments, the CTSL inhibitor may be specific for cathepsin L, cathepsin B and cathepsin S. In still other embodiments, the CTSL inhibitor is Vitamin D.

A DNA-damaging agent refers to a chemical compound that promotes DNA damage and is useful in the treatment of cancer. Suitable DNA-damaging agents are well known in the art. Non-limiting examples of DNA-damaging agents may include an alkylating agent, an anti-metabolite, a topoisomerase inhibitor, and a PARP inhibitor. A skilled practitioner will be able to determine the appropriate dose of the DNA-damaging agent.

Suitable alkylating agents include, but are not limited to, altretamine, benzodopa, busulfan, carboplatin, carboquone, carmustine (BCNU), chlorambucil, chlornaphazine, cholophosphamide, chlorozotocin, cisplatin, cyclosphosphamide, dacarbazine (DTIC), estramustine, fotemustine, ifosfamide, improsulfan, lipoplatin, lomustine (CCNU), mafosfamide, mannosulfan, mechlorethamine, mechlorethamine oxide hydrochloride, melphalan, meturedopa, mustine (mechlorethamine), mitobronitol, nimustine, novembichin, oxaliplatin, phenesterine, piposulfan, prednimustine, ranimustine, satraplatin, semustine, temozolomide, thiotepa, treosulfan, triaziquone, triethylenemelamine, triethylenephosphoramide (TEPA), triethylenethiophosphaoramide (thiotepa), trimethylolomelamine, trofosfamide, uracil mustard and uredopa.

Non-limiting examples of suitable anti-metabolites include aminopterin, ancitabine, azacitidine, 8-azaguanine, 6-azauridine, capecitabine, carmofur (1-hexylcarbomoyl-5-fluorouracil), cladribine, clofarabine, cytarabine (cytosine arabinoside (Ara-C)), decitabine, denopterin, dideoxyuridine, doxifluridine, enocitabine, floxuridine, fludarabine, 5-fluorouracil, gemcetabine, hydroxyurea (hydroxycarbamide), leucovorin (folinic acid), 6-mercaptopurine, methotrexate, nafoxidine, nelarabine, oblimersen, pemetrexed, pteropterin, raltitrexed, tegofur, tiazofurin, thiamiprine, tioguanine (thioguanine), and trimetrexate.

Suitable topoisomerase inhibitors include, but are not limited to, amsacrine, etoposide (VP-16), irinotecan, mitoxantrone, RFS 2000, teniposide, and topotecan.

Non-limiting examples of PARP inhibitors include iniparib, olaparib, rucaparib, veliparib, CEP 9722, MK 3827, BMN-673, and 3-aminobenzamide.

As used herein, the term “DNA-damaging agent” also refers to radiation therapy. Radiation therapy may include, but is not limited to, the use of X-rays, gamma-rays, and/or the directed delivery of radioisotopes to tumor cells. Other forms of DNA damaging factors are also contemplated, such as microwaves and UV-irradiation. Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend upon the half-life of the isotope, the strength and type of radiation emitted, and the uptake by the neoplastic cells.

Contacting the tumor cell with an effective amount of a CTSL inhibitor and a DNA-damaging agent generally involves adding the CTSL inhibitor and a DNA-damaging agent to the tumor cell. This may occur in vitro, ex vivo, or in vivo. When the tumor cell comprises a breast tumor in a subject, the CTSL inhibitor and a DNA-damaging agent may come into contact with the tumor cell after administration of the CTSL inhibitor and a DNA-damaging agent to the subject.

The term “effective amount”, as used herein, means an amount of a substance such as a compound that leads to measurable and beneficial effects for the subject administered the substance, i.e., significant efficacy. The effective amount or dose of compound administered according to this discovery will be determined by the circumstances surrounding the case, including the compound administered, the route of administration, the status of the symptoms being treated and similar patient and administration situation considerations among other considerations. The frequency of dosing may be daily or once, twice, three times or more per week or per month, as needed as to effectively treat the symptoms.

The CTSL inhibitor and the DNA damaging agent may each be administered orally, parenterally (including subcutaneous injections, intravenous, intramuscular, intrasternal injection or infusion techniques), by inhalation spray, buccal or sublingual, in the form of a unit dosage of a pharmaceutical composition containing an effective amount of the compound and conventional non-toxic pharmaceutically-acceptable carriers, adjuvants and vehicles. Typical dosage levels can be determined and optimized using standard clinical techniques and will be dependent on the mode of administration.

The term “administration” and variants thereof (e.g., “administering”) in reference to a CTSL inhibitor or a DNA damaging agent mean providing the CTSL inhibitor, the DNA damaging agent, or a prodrug thereof to the subject in need of treatment, and is understood to include concurrent and sequential provision of the CTSL inhibitor, the DNA damaging agent, or prodrug thereof.

By “pharmaceutically acceptable” is meant that the ingredients of the pharmaceutical composition must be compatible with each other and not deleterious to the recipient thereof.

The pharmaceutical compositions may be in the form of orally-administrable suspensions or tablets or capsules, nasal sprays, sterile injectible preparations, for example, as sterile injectible aqueous or oleagenous suspensions or suppositories. These compositions are deliberately designed to be appropriate for the selected mode of administration, and pharmaceutically acceptable excipients such as compatible dispersing agents, buffers, surfactants, preservatives, solubilizing agents, isotonicity agents, stabilizing agents and the like are used as appropriate. Remington's Pharmaceutical Sciences, Mack Publishing Co., Easton Pa., 16Ed ISBN: 0-912734-04-3, latest edition, incorporated herein by reference in its entirety, provides a compendium of formulation techniques as are generally known to practitioners.

### III. Methods for Predicting the Effectiveness of a DNA Damaging Agent in Reducing Tumor Growth

Another aspect of the invention provides a method for predicting the effectiveness of a DNA damaging agent in reducing tumor growth in a subject in need thereof. Typically, the method comprises obtaining a sample of a breast tumor from a subject, processing the sample in vitro to determine the levels of nuclear CTSL and nuclear 53BP1 in at least one tumor cell comprising the sample, classifying the tumor as non-responsive to a DNA-damaging agent if the sample has (i) high nuclear CTSL and low nuclear 53BP1 or (ii) low nuclear CTSL and low nuclear 53BP1, and identifying a subject with a tumor classified as non-responsive to a DNA-damaging agent as a subject for which a DNA-damaging agent would not be effective.

Methods of processing the sample in vitro to determine the levels of nuclear CTSL and nuclear 53BP1 are described above. In some embodiments, the method is selected from the group consisting of immunohistochemistry, flow cytometry, array and ELISA. In other embodiments, the method is mass spectrometry. Suitable DNA-damaging agents, breast tumors, samples, subjects and levels are also described above.

In some embodiments, if the sample has high nuclear CTSL and low nuclear 53BP1, the subject may be administered CTSL inhibitor to increase the effectiveness of a DNA-damaging agent. Suitable CTSL inhibitors, and methods for administering thereof, are described above.

### EXAMPLES

The following examples illustrate various iterations of the invention.

### Material and Methods for Examples 1-7

Cell Culture:

MCF7 cells were a gift from J. Weber (Washington University, St. Louis, Mo.). Cells were maintained in DMEM (Cellgro) supplemented with 10% fetal bovine serum (Sigma-Aldrich), antibiotics, and antimycotics. Cells transduced with short hairpin RNAs (shRNAs) were maintained in selection media containing 0.5 mg/mL Geneticin G418 (Gibco) or 2 μg/mL puromycin (Invitrogen).

Immunoblotting:

Cells were lysed in RIPA buffer (50 mM Tris-HCl pH 8.0, 150 mM NaCl, 1% NP-40, 0.5% sodium deoxycholate, 0.5% SDS) supplemented with PMSF, protease inhibitors, and DTT. Following sonication, 120 μg of total cell lysate was loaded into 4%-15% Tris-Gly gradient gels. Protein detection was carried out with the following antibodies: BRCA1 (sc-6954, Santa Cruz), 53BP1 (NB100-304, Novus Biologicals), cathepsin L (sc-6498, Santa Cruz), p107 (sc-318, Santa Cruz), RAD51 (sc-8349, Santa Cruz), RPA (NA18, Calbiochem), and β-Tubulin (T8328, Sigma-Aldrich).

Proliferation Assay:

Cells were plated in triplicate at 150,000 cells per well in 6 well plates and counted 96 hours later. Cell proliferation was measured in each cell line a total of 4 times within a 14 day period. In order to extrapolate proliferation to a 14 day period, the equation N=N0ekt was used where N is the final number of cells, N0 is the starting number of cells, k is In 2/DT (doubling time), and t is time in days (Sherley et al, 1995). For each 96 hour period the doubling time was calculated and used to estimate the number of cells (N) that would result from initially plating 150,000 (N0) and culture them for a given period of time. The doubling times for the control cells remained constant throughout the time period, while the doubling times for the sh BRCA1 cells started increasing once the cells overcame the growth arrest.

Quantitative Reverse-Transcription PCR:

RNA was isolated using the RNAqueous-4PCR Kit (Ambion) following manufacturer's instructions. cDNA was generated from 500 ng of total RNA using TaqMan Reverse Transcription Reagents (Applied Biosystems). BRCA1, 53BP1, cathepsin L and 18S expression was determined using TaqMan Gene Expression Assays (Hs01556193_m1, Hs00996818_m1, Hs00377632_m1, Hs99999901_s1, respectively, Applied Biosystems). For the analysis, all reactions (in triplicate) were carried out by amplifying target gene and endogenous controls in the same plate. Relative quantitative evaluation of target gene was determined by comparing the cycle thresholds.

Constructs and Viral Transduction:

The shRNA targeting BRCA1 was a gift from Junran Zhang (Case Western Reserve University, Cleveland, Ohio). The shRNAs targeting cathepsin L were purchased from Sigma-Aldrich. Short hairpin controls were either purchased from Sigma-Aldrich or were gifts from Junran Zhang or Sheila Stewart (Washington University, St. Louis, Mo.). Lentiviral transductions were performed as previously described (Gonzalez-Suarez et al., 2009). Briefly, 293T cells were transfected using Fugene (Roche) with viral packaging and envelope plasmids (pHR'8.2ΔR and pCMV-VSV-G, respectively, provided by Sheila Stewart, Washington University) along with the appropriate vector containing the shRNA of interest. Forty eight hours post transfection, virus containing media was collected and used to infect target cells. Transductions were carried out in one 4 hour infection. Cells were allowed to recover for 48 hours followed by treatment with the appropriate selection drug.

Treatment with Vitamin D:

All treatments with vitamin D were performed by incubating cells with 10−7 M 1α,25-dihydroxyvitamin D3 (Calcitriol, D1530, Sigma-Aldrich). Aliquots of 1 nmol calcitriol were resuspended in 1 mL of bovine growth serum (BGS) and diluted in DMEM with antibiotics and antimycotics to a final concentration of 10% BGS. BGS was used as the vehicle in control media. Treatment was carried out for 24 hours unless indicated otherwise.

Treatment with E-64:

Cells were incubated with the broad spectrum cathepsin inhibitor E-64 (E3132, Sigma-Aldrich) at a concentration of 10 μM. Water served as the vehicle in control media. Treatment was carried out for 24 hours unless indicated otherwise.

Treatment with PARPi:

Cells were treated with the PARPi EB-47 (E8405, Sigma-Aldrich) at a concentration of 1.2 μg/mL for 48 hours. PARPi was dissolved in water, which was used as vehicle control.

Treatment with Ionizing Radiation:

For determining the extent of genomic instability, cells were irradiated with 2 Gy and analysis of metaphase spreads was performed 24 hours post-irradiation. For assaying formation of IRIF, cells were irradiated with 8 Gy and fixed and processed for immunofluorescence 1 hour, 3 hours, or 6 hours post-irradiation as indicated. For comet assays, cells were irradiated with 8 Gy.

Immunofluorescence:

Cells were plated on coverslips, irradiated with 8 Gy if indicated, and collected at the varying time intervals. Cells were fixed with 3.7% formaldehyde and 0.2% TritonX-100 in PBS for 10 minutes at RT, followed by 3 washes in PBS. After blocking in 1% BSA and 0.1% Triton X-100 in PBS for 1 hour at 37° C., cells were incubated with 53BP1 (NB100-304, Novus Biologicals) or RAD51 (sc-8349, Santa Cruz) antibodies for 1 hour at 37° C. Three washes in PBS were carried out followed by incubation for 1 hour at 37° C. with appropriate secondary antibodies. Final washes in PBS were carried out and cells were counterstained with DAPI and coverslips were mounted on slides using Vectashield (Vector Labs). Fluorescent micrographs were taken using a Nikon 90i upright microscope.

Comet Assay:

Neutral comet assays were performed using CometSlide assay kits (Trevigen). Control (sh scr) and BOGA cells (sh BRCA1) treated with vehicle or vitamin D for 24 h were irradiated with 8 Gy and incubated at 37° C. for different periods of time (0, 30, 60, and 90 minutes) to allow for DNA damage repair. Cells were embedded in agarose, lysed, and subjected to neutral single cell gel electrophoresis. The agarose was dehydrated and cells were stained with ethidium bromide to visualize under a fluorescence microscope. Olive comet moment was determined using the program CometScore Version 1.5 (TriTek). Olive moment was calculated by multiplying the percentage of DNA in the tail by the displacement between the means of the head and tail distributions as described (Olive et al., 1990). A total of 25-30 comets were analyzed per sample in each experiment.

Analysis of Aberrant Chromosomes:

Cells were treated with vitamin D or E-64 for 24 hours, irradiated, and allowed to recover for 24 hours. Cells treated with PARPi were pre-treated with vitamin D for 24 hours followed by combined treatment with vitamin D and PARPi for 48 hours. Following all treatments, cells were arrested in mitosis by treatment with colcemid for 4 hours and metaphase spreads prepared by hypotonic swelling in 0.56% KCl, followed by fixation in 3:1 methanol:acetic acid. Cell suspensions were dropped onto slides and stained for 25 minutes in Wright-Giemsa Stain (9380-32, Ricca Chemical Company) and then washed in water. Slides were allowed to dry and were mounted using Eukitt Mounting Reagent and analyzed on an Olympus BX51 light microscope.

Flow Cytometry:

Cells were collected, washed in PBS and fixed in 70% ethanol. To determine DNA content, cells were stained for 15 min at 37° C. followed by staining with 500 μg/μL propidium iodide in PBS containing 0.1% Triton-X 100 and 20 μg/mL RNAse A. Data were collected using a FACScan flow cytometer. For each sample, 10,000 events were collected, and aggregated cells were gated out.

Tissue Tumor Microarrays (TMA):

A total of 180 tissue samples from patients with breast carcinoma were obtained at Hospital Universitari Arnau de Vilanova in Lleida, Spain from 1998 to 2010 before the initiation of neoadjuvant treatment. An informed consent was obtained from each patient, and the study was approved by the local Ethical Committee. The series of 180 tumor samples included formalin-fixed, paraffin-embedded blocks for all patients, 155 core biopsies, and 25 surgical specimens. Tumors were classified according to the expression of the following proteins: Ki67, ERα and Her2 into four molecular subtypes: Luminal A (n=70), Luminal B (n=46), ERBB2 (n=36), Triple negative (n=28). A Tissue arrayer device (Beecher Instrument, MD) was used to construct the TMA. Briefly, all samples were histologically reviewed and representative areas were marked in the corresponding paraffin-blocks. Two selected cylinders (0.6 mm of largest diameter) from two different areas were included for each case.

Immunohistochemical Analysis:

TMA blocks were sectioned at a thickness of 3 μm, dried for 1 h at 65° C. before being dewaxed in xylene and rehydrated through descending concentrations of ethanol, and washed with phosphate buffered saline. Ki67, ERα and HER2 were used to determine molecular subtype. Comparative studies of CTSL and 53BP1 expression were carried out on sequential serial sections. Antigen retrieval for CTS L and ERα was achieved by heat treatment at 95° C. for 20 min in a high pH solution (DAKO). Heat-induced antigen retrieval for 53BP1 and Ki67 was performed in a low pH solution (DAKO). Before staining the sections, endogenous peroxidase was blocked. Primary antibodies and incubation times were as follows: CTSL (1:50; S-20, Santa Cruz Biotechnology, incubation overnight at 4° C.); 53BP1 (1:2500; NB100-304, Novus Biologicals, incubation 20 minutes at room temperature); Ki67 (Ready-to-use; M1B, DAKO, incubation 20 minutes at room temperature); ERα (Ready-touse; 1D5, DAKO, incubation 20 minutes at room temperature), and HER2 (Herceptest Kit, DAKO). The reaction was visualized with the Streptovidin-Biotin Complex (DAKO) for CTSL and Envision Flex (DAKO) for 53BP1, Ki67 and ERα. Sections were counterstained with haematoxylin. Appropriate positive and negative controls were also tested.

Immunohistochemical scores provided a semiquantitative measurement of protein expression for each tumor, taking into consideration the percentage and intensity of the staining, and the percentage of positive cells. A histological score ranging from 0 (no immune reaction) to 300 (maximal immunoreactivity) was obtained with the formula Histoscore (Hscore)=1×(% light staining)+2×(% moderate staining)+3×(% strong staining). The reliability of such scores for the interpretation of immunohistochemical staining in TMAs has been reported (Pallares et al., 2009). Nuclear Hscore for cathepsin L and 53BP1 were chosen as cut-off points for their maximal power to discriminate between different molecular tumor types for these antibodies. Cut off points were Hscores of 50 for nuclear cathepsin L, and of 115 for nuclear 53BP1.

Her2 staining was evaluated according to a standard protocol (HercepTest; DAKO) and scored as 4 intensities (i.e. negative=0; weak=1+; moderate=2+; and strong=3+), considering negative HER2 expression for intensity values of 0, 1+ and 2+ when there was no amplification by FISH, and positive for intensity values of 3+ and 2+, when 2+ was amplified by FISH. For each marker, there was a variable number of non-assessable cases due to technical problems including no representative tumor sample left in the cylinders, detachment, cylinders missed while constructing the array, necrosis, and absence of viable tumor cells in the TMA sections.

The reproducibility of TMA immunostaining for each antibody was confirmed by comparing TMA's results with those obtained in sections from the corresponding paraffin blocks in 20 randomly selected cases using the Proof of Wilcoxon and Pearson Linear Correlation.

Statistical Analysis:

For the in vitro experiments with MCF7 cells, a “two-tailed” student's t-test was used to calculate statistical significance of the observed differences. Microsoft Excel v.2010 was used for the calculations. In all cases, differences were considered statistically significant when p<0.05. For the TMA studies, Fisher Exact Test assessed the statistical significance of the differences in cathepsin L and 53BP1 Hscores between molecular breast tumor subtypes. The Spearman correlation assessed the significance of the relationship between nuclear CTSL and 53BP1 Hscores for all breast tumor types.

### Example 1

### BRCA1 Depletion Leads to Upregulation of CTSL and Degradation of 53BP1

Previous studies revealed an important role for the cysteine protease CTSL in regulating the levels of 53BP1 protein in fibroblasts (Gonzalez-Suarez et al., 2011; Redwood et al., 2011). Given the recent association between BRCA1 loss of function and decreased levels of 53BP1 in breast tumor cells, it was investigated whether these two processes are functionally related. In particular, it was determined whether CTSL is one of the factors contributing to 53BP1 loss in BRCA1-deficient cells. The breast cancer cell line MCF7, which is proficient in both BRCA1 and 53BP1, was lentivirally transduced with a shRNA specific for depletion of BRCA1 or a shRNA control (FIG. 1A). Depletion of BRCA1 induced an initial growth arrest in MCF7 cells while control cells continued growing exponentially (FIG. 1B). Moreover, growth arrested BRCA1-deficient cells did not show differences in the levels of 53BP1 or CTSL proteins when compared to control cells (FIG. 1C). Interestingly, after 7 to 14 days in culture (depending on the experiment), BRCA1 deficient cells resumed proliferation, although at a lower rate than BRCA1-proficient cells (FIG. 1D). Thus, an in vitro model to study molecular mechanisms that contribute to proliferation and survival of BRCA1 deficient cells was established. Importantly, BRCA1-deficient cells that Overcome Growth Arrest (herein referred to as BOGA cells, for clarity) exhibit a marked decrease in 53BP1 protein levels (FIG. 1E), recapitulating the phenotype of breast cancer cells carrying loss of function mutations in BRCA1. In addition, these cells exhibit a marked increase in CTSL protein levels, especially in the active form of the protease (FIG. 1E). Importantly, activation of CTSL and downregulation of 53BP1 protein levels were also observed in MDA-MB-231 breast cancer cells that overcome the growth arrest induced by depletion of BRCA1, indicating that this pathway is not specific of MCF7 cells (FIG. 2A).

To determine if BRCA1 regulates the levels of these proteins at the transcriptional level in MCF7 cells, transcripts levels of CTSL and 53BP1 were monitored by qRT-PCR. Depletion of BRCA1 led to transcriptional upregulation of CTSL, without significant differences in 53BP1 transcripts levels (FIG. 1F). Given the known role of CTSL in the degradation of 53BP1, these results suggest that upregulation of CTSL upon loss of BRCA1 could be responsible for the decrease in 53BP1 protein levels, which in turn would allow the survival and proliferation of BRCA1-deficient cells. Previous studies had shown that subsets of BRCA1-mutated breast tumors exhibit a marked reduction in 53BP1 mRNA levels (Bouwman et al., 2010). However, these results show that acute loss of BRCA1 can also lead to downregulation of 53BP1 protein levels, indicating that different mechanisms are activated in BRCA1-deficient cells to lower 53BP1 levels and ensure cell survival.

### Example 2

### CTSL is Responsible for Degradation of 53BP1 Following Depletion of BRCA1

To test if CTSL is responsible for the degradation of 53BP1, acute depletion of CTSL was performed in control and BOGA cells (FIG. 3). The decrease in CTSL transcripts levels in both cell lines upon lentiviral transduction with a specific shRNA is shown in FIG. 2B. Good depletion of CTSL protein levels was achieved in control and BOGA cells (FIG. 2A). Intriguingly, a slight increase in BRCA1 protein levels in BOGA cells depleted of CTSL was observed, suggesting a putative feedback mechanism of CTSL on BRCA1 protein levels. Importantly, depletion of CTSL stabilized 53BP1 protein levels in BOGA cells mirroring those of control cells (FIG. 2B). As a control, it is shown that transcripts levels of BRCA1 are not affected by depletion of CTSL (FIG. 2C). These data indicate that cells that bypass the growth arrest imposed by the loss of BRCA1 activate CTSL-mediated degradation of 53BP1.

Previous studies demonstrated that treatment with vitamin D inhibits CTSL activity and stabilizes 53BP1 protein levels in MEFs (Gonzalez-Suarez et al., 2011), putatively via upregulation of cystatins, endogenous inhibitors of cathepsin activity (Gonzalez-Suarez et al., 2011). In addition, it has been shown that CTSL degrades Rb family members pRb and p107 (Redwood et al., 2011). Here, it is shown that treatment of BOGA cells with vitamin D stabilizes the levels of 53BP1 (FIG. 3C). The stabilization of p107 by vitamin D treatment was used as control for vitamin D inhibition of CTSL activity. Similarly, treatment of BRCA1-deficient cells with the cathepsin inhibitor E64 leads to increased levels of 53BP1 protein (FIG. 3D). Note how depletion of BRCA1 leads to upregulation of CTSL levels and how treatment with the inhibitor E64 stabilizes 53BP1 without major changes in CTSL levels, indicating that it functions by inhibiting activity. Altogether, these results demonstrate that transcriptional upregulation of CTSL upon loss of BRCA1 leads to degradation of 53BP1 and that inhibition of CTSL stabilizes the levels of 53BP1 protein.

### Example 3

### CTSL-Mediated Degradation of 53BP1 Rescues HR Defects in BRCA1-Deficient Cells

CTSL-mediated degradation of 53BP1 rescues HR defects in BRCA1-deficient cells The role of BRCA1 in the maintenance of homology-mediated repair has been clearly demonstrated (Moynahan et al., 1999; Moynahan et al., 2001; Scully et al., 1997; Scully et al., 1996; Snouwaert et al., 1999; Westermark et al., 2003). In particular, BRCA1 promotes end-resection by CtIP and the MRN complex at DSBs, an event required for efficient HR. Thus, BRCA1-deficiency impairs the localization of RAD51 protein at DSBs following ionizing radiation (IR) treatment. Interestingly, recent studies demonstrated that loss of 53BP1 in BRCA1-deficient cells partially rescues HR and thus the localization of RAD51 at IR-induced foci (IRIF) (Bunting et al., 2010). Based on these data, it was tested whether CTSL-mediated degradation of 53BP1 in BOGA cells might inhibit 53BP1 foci formation and promote DNA DSBs repair by HR. Also tested was whether inhibition of CTSL could rescue 53BP1 recruitment to DSBs and reduce HR. To test these points, the formation of 53BP1 and RAD51 IRIF immunofluorescence were monitored in control and BOGA cells at different post-irradiation times and following treatment with vitamin D or vehicle. First, it was demonstrated that MCF7 cells growth arrested due to depletion of BRCA1 retained their ability to recruit 53BP1 protein to IRIF (FIG. 4A), consistent with the normal levels of 53BP1 observed in these cells (FIG. 1C). In contrast, BOGA cells are unable to form 53BP1 IRIF, consistent with the global decrease in 53BP1 protein levels (FIGS. 4B, 1E, and 5, and Table 1). Next, it was determined if the deficiency in 53BP1 foci formation could be rescued by inhibition of CTSL via treatment with vitamin D. BOGA cells were treated with vitamin D or vehicle and the formation of 53BP1 IRIF was compared to control cells. As shown in FIGS. 4C, 4D and 5, and Table 1, BOGA cells treated with vehicle present with defects in the formation of 53BP1 IRIF (27% cells positive versus 83% positive control cells). Interestingly, treatment with vitamin D rescued 53BP1 foci formation (84% positive cells). These results demonstrate that inhibition of CTSL by vitamin D rescues the levels of 53BP1 and the recruitment of the protein to DSBs in the absence of BRCA1.

Next, the formation of RAD51 IRIF in BOGA cells and BRCA1-proficient control cells were monitored at different times post-irradiation. In contrast to 53BP1, RAD51 foci formation was indistinguishable between control and BOGA cells at 1 h post-irradiation (FIGS. 6A and 6B). These data suggest that activation of CTSL-mediated degradation of 53BP1 allows end-resection of DSBs and RAD51 foci formation in the absence of BRCA1. Based on the theory of competition between repair pathways—NHEJ and HR—for the repair of DSBs, it was hypothesized that stabilization of 53BP1 in BOGA cells could inhibit end-resection at the breaks and thus reduce the extent of RAD51 foci formation. To test this, BOGA cells were treated with vitamin D or vehicle for 48 h followed by radiation and monitoring of the formation of RAD51 IRIF. Interestingly, treatment with vitamin D was found to reduce the formation of RAD51 foci in the context of BRCA1-deficiency (from 56% in vehicle treated cells to 35% in vitamin D treated cells) (FIGS. 6A and 6B), revealing an unprecedented role for vitamin D in modulating the extent of HR in breast cancer cells.

Intriguingly, it was found that while control cells retain the ability to form RAD51 IRIF for up to 6 hours post-irradiation, the percentage of BOGA cells positive for RAD51 IRIF decreased significantly with time post-irradiation, such that a reduction of approximately 40% was observed 3 h post-IR and of 80% at 6 h post-IR (FIGS. 6C and 7). These results indicate that 53BP1 deficiency in BOGA cells allows formation of RAD51 foci at early times post-irradiation. However, the absence of BRCA1 results in deficiencies in the recruitment or maintenance of RAD51 protein at DSBs over time. Interestingly, the stabilization of 53BP1 by vitamin D did not affect the extent of RAD51 IRIF at later times post-irradiation (3 h), suggesting that these later events might be independent of 53BP1 levels.

Overall, these studies demonstrate that activation of CTSL-mediated degradation of 53BP1 rescues to certain extent the HR defects associated with BRCA1 loss, and that inhibition of CTSL activity renders cells unable to properly perform HR. These findings provide a novel strategy to modulate HR efficiency in BRCA1 deficient cells which could be exploited with therapeutic purposes.

TABLE 1
CTSL-mediated degradation of 53BP1 in BRCA1-deficient cells

| | | sh BRCA1 | sh BRCA1 |
|---|---|---|---|
| | sh scr | vehicle | vit D |
| Cell number | 1084 | 1498 | 1215 |
| % cells with 53BP1 foci | 83% | 27% | 84% |

sh scr = BOGA cells treated with sh RNA scramble
sh BRCA1 vehicle = BOGA cells treated with sh RNA targeting BRCA1 + vehicle
sh BRCA1 vit D = BOGA cells treated with sh RNA targeting BRCA1 + vitamin D

### Example 4

### Consequences of CTSL-Mediated Degradation of 53BP1 for DNA Repair and Genomic Stability

To determine how CTSL-mediated degradation of 53BP1 affects the kinetics of DNA DSBs repair in BRCA1-deficient cells, we performed neutral comet assays (Olive et al., 1990). The repair of DNA DSBs usually follows bimodal kinetics with a fast phase of repair associated with classical NHEJ, and a slower phase of repair typically associated with HR or alternative NHEJ (Iliakis et al., 2004). The results in FIG. 8A show that BOGA cells that have activated CTSL-mediated degradation of 53BP1 exhibit defects in the fast phase of repair corresponding to classical NHEJ. Furthermore, FIG. 8A shows that inhibition of CTSL via treatment with vitamin D rescues the kinetics of DNA DSBs repair, mirroring that of control cells. These results suggest that CTSL-mediated degradation of 53BP1 hinders NHEJ in BRCA1-deficient cells. However, these cells are still able to repair DSBs despite at a lower rate, suggesting that repair by HR or alternative-NHEJ might remain relatively intact. This is consistent with findings that RAD51 foci are able to form at early times post-irradiation. Importantly, inhibition of CTSL by vitamin D can rescue the fast phase of repair.

Cells deficient in HR become dependent on alternative DNA DSBs repair pathways which often join breaks from different chromosomes leading to complex chromosomal aberrations that if severe, result in cell death. Previous studies demonstrated that loss of 53BP1 reduces the extent of chromosomal aberrations or genomic instability characteristic of BRCA1-deficient cells (Bunting et al., 2010). This rescue was proposed to be due to a decrease in 53BP1-mediated NHEJ. To test whether stabilization of 53BP1 in BRCA1-deficient cells would exacerbate the extent of genomic instability after IR, genomic instability was monitored in control and BOGA cells by analyzing chromosomal aberrations in metaphase spreads 24 hours after IR. No profound genomic instability was found after irradiation in BOGA cells (FIG. 8B), in agreement with the decrease in both BRCA1 and 53BP1 and thus the ability to repair the damage generated, despite at a lower rate. However, stabilization of 53BP1 in this context by treatment with vitamin D significantly increased the percentage of metaphases with aberrant chromosomes after IR. Similarly, stabilization of 53BP1 in BOGA cells by treatment with the cathepsin inhibitor E64 (FIG. 3D), leads to a marked increase in genomic instability after IR (FIG. 8C). These results demonstrate that the extent of CTSL-mediated degradation of 53BP1 is a key determinant of the ability of BRCA1-deficient cells to deal with the amount of DNA damage generated by IR and putatively other genotoxic agents. Consistent with the increase in genomic instability, treatment of BOGA cells with vitamin D significantly reduced the recovery from IR (FIG. 8D). Thus, inhibition of CTSL could represent a novel strategy to induce radiation sensitivity in specific types of breast tumors.

A number of studies have demonstrated that BRCA1-deficient cells are exquisitely sensitive to PARPi, drugs at the forefront for breast cancer treatment (Bryant et al., 2005; Drew et al., 2010). The reason behind this effect is that PARPi prevent repair of ssDNA breaks, which are converted to DSBs during replication. The inability of HR-deficient cells to properly repair these breaks leads to high levels of genomic instability and cell death (Farmer et al., 2005). Importantly, loss of 53BP1 in the context of BRCA1 deficiency was shown to rescue HR and genomic stability, reducing the sensitivity of these cells to PARPi (Aly and Ganesan, 2011; Bunting et al., 2010). As shown in FIG. 8E, treatment of BOGA cells with PARPi does not result in profound genomic instability, in agreement with the resistance of cells double deficient in 53BP1 and BRCA1 to this treatment. Interestingly, stabilization of 53BP1 by vitamin D results in an increase in the extent of chromosomal aberrations in response to PARPi.

These results indicate that activation of CTSL-mediated degradation of 53BP1 contributes to the resistance of BRCA1-deficient cells to PARPi. Thus, strategies that stabilize 53BP1, i.e. vitamin D or CTSL inhibitors, could represent promising strategies for the treatment of BRCA1-mutated cancers, especially those that upregulate CTSL as a means to lower 53BP1 protein levels. The combined inhibition of cathepsin and PARP activities could have a positive outcome for patients carrying BRCA1-mutated tumors.

### Example 5

### Inverse Correlation Between CTSL and Levels of 53BP1 and BRCA1 During the Cell Cycle

BRCA1 expression increases as cells progress through the G1 phase of the cell cycle, peak during S phase, and remain elevated at the G2/M transition (Vaughn et al., 1996). No information is available to date about how the levels of 53BP1 are regulated during the cell cycle. However, the fact that NHEJ occurs at all phases has led to the notion that 53BP1 levels might be constant throughout the cell cycle. Based on the finding that CTSL participates in the degradation of 53BP1, it was investigated whether there is a relationship between CTSL and 53BP1 levels during the cell cycle. Human fibroblasts (BJ) immortalized with telomerase (BJ+hTert) were used because they have intact cell cycle regulation and undergo growth arrest by contact inhibition, obviating the need for drugs used to synchronize cells that cause DNA damage. Cells arrested in G0/G1 phases for 48 h were plated at diluted concentrations and allowed to progress through the cycle. The cell cycle profile and the levels of 53BP1 and CSTL were monitored at different times after the release from growth arrest and compared to those of asynchronously growing cells (FIG. 9). Arrest in G0/G1 was found to lead to a marked increase in the levels of active CTSL and a decrease in 53BP1 levels (FIGS. 9A and 6B, 0 h). A decrease in BRCA1 levels was also observed in this phase, as previously shown (Vaughn et al., 1996). Furthermore, as cells progress through the cycle, a progressive reduction in CTSL levels was observed which correlated with upregulation of 53BP1 and BRCA1. The average of the levels of all these proteins in three independent experiments is shown in FIG. 9C. As control, the levels of the repair factor RPA were monitored, which remain constant throughout the cycle, and the levels of RAD51 which change during the cycle. These studies demonstrate that the levels of 53BP1 fluctuate during the cell cycle, mirroring the expression of BRCA1. In addition, the levels of active CTSL are inversely correlated with the levels of both proteins, suggesting that BRCA1 might regulate the levels of 53BP1 during the cell cycle via CTSL.

To determine whether CTSL contributes to the decrease in 53BP1 in G0/G1 arrested cells, lentiviral transduction of BJ+hTert fibroblasts with a shRNA specific for CTSL depletion or a shRNA control were performed (FIG. 9D). Following selection of infected cells, fibroblasts were arrested in G0/G1 by growing them to confluency. After 48 h of growth arrest, cells were collected and the levels of 53BP1, BRCA1 and CTSL were monitored by western blot. Growth arrested cells that are depleted of CTSL exhibited higher levels of 53BP1 than CTSL-proficient cells. These results indicate that CTSL plays a role in the downregulation of 53BP1 in growth arrested cells. Interestingly, depletion of CTSL led to an increase in the levels of BRCA1 as well, supporting the notion of a feedback mechanism of CTSL on BRCA1 protein levels. Overall, the data indicate that CTSL plays a role in the regulation of 53BP1 and putatively BRCA1 during the cell cycle. In particular, decreases in BRCA1 in G0/G1 phases of the cycle might lead to transcriptional activation of CTSL and degradation of 53BP1 and possibly BRCA1. At subsequent stages of the cell cycle, the upregulation of BRCA1 would reduce CTSL-mediated degradation of 53BP1 stabilizing the levels of the protein. Altogether the data demonstrates an unprecedented role for CTSL in the regulation of 53BP1 protein levels in normal and tumor cells.

### Example 6

### Increased Levels of Nuclear CTSL in TNBC

CTSL, one of the most abundant proteases in the endosomal/lysosomal compartment, has recently been identified in the nucleus, where it processes in a regulated manner the N-terminal tail of histone H3 and the transcription factor CDP/Cux (Duncan et al., 2008; Goulet et al., 2004). In addition, it has been previously demonstrated that upregulation of CTSL in fibroblasts leads to accumulation of the protease in the nucleus and degradation of 53BP1 protein (Gonzalez-Suarez et al., 2011). Recent studies in two cohorts of breast tumors (Yale cohort of 444 tumors and Helsinki cohort of 1187 tumors) demonstrated that loss of 53BP1 is more frequent in triple-negative and BRCA1/BRCA2 mutated breast cancers (Bouwman et al., 2010). In light of these findings, it was tested whether upregulation of CTSL is a common event in human breast cancers of the poorest prognosis and if it correlates with decreased levels of 53BP1.

Analyses of multitumor tissue microarrays (TMA) constructed with tissue from 180 patients' biopsies and lumpectomies were performed by immunohistochemistry (IHC), as shown in FIG. 10. Tumors were classified according to the expression of Ki67, ERα and Her2 into four molecular subtypes: Luminal A (n=70), Luminal B (n=46), ERBB2 (n=36), Triple-Negative (n=28). Luminal A tumors are steroid hormone receptor—positive, negative for HER2 and contain less than 30% of Ki67 positive cells, and tend to have a good prognosis. Luminal B tumors are steroid hormone receptor—positive, negative for HER2 and contain more than 30% Ki67 positive cells; and tend to have a worse prognosis than luminal A. In contrast, ERBB2 tumors are positive for HER2 and have been shown to have a poor prognosis. Triple-Negative tumors are negative for steroid hormone receptors and HER2 and have the worst prognosis.

Immunohistochemical scores (Hscores) for Ki67, ER, cathepsin-L and 53BP1 provided a semiquantitative measurement of their expression for each tumor, taking into consideration the percentage and intensity of the staining, and also the percentage of positive cells. The reliability of such scores for the interpretation of immunohistochemical staining in TMAs has been reported (Pallares et al., 2009). As shown in FIG. 10, immunohistochemical staining of cathepsin L was both cytoplasmic and nuclear; HER2 labeling was clearly at the plasma membrane; and Ki67, ER and 53BP1 expression was only nuclear. Nuclear Hscores for CTSL and 53BP1 were used to calculate the cut-off points for each antigen, with maximal power to discriminate between different molecular tumor types. Cut off points were Hscores of 50 for nuclear CTSL, and of 115 for nuclear 53BP1.

Table 2 summarizes the immunohistochemical results. Whereas cytoplasmic CTSL Hscores were similar in all tumor subtypes, nuclear CTSL Hscores were markedly enhanced in Triple-Negative tumors (p=0.003). Furthermore, 10 out of 27 cases of Triple-Negative tumors (37%) elicited an Hscore for nuclear CTSL above the identified cut off of 50. In contrast, only 4 cases out of 145 (4%) of the rest of molecular tumor subtypes expressed nuclear CTSL above 50 (p=0.000006). The nuclear 53BP1 Hscores in Triple-Negative tumors were lower than in all other tumor subtypes (p=0.0009). Using the cut-off value of 115 for 53BP1, 10 cases out of 28 (35.7%) of Triple-Negative tumors expressed 53BP1 Hscore 115 compared to 55 cases out of 70 (78.6%) for Luminal A tumors, 36 cases out of 46 (80.4%) for luminal B, and 22 cases out of 32 (68.8%) in Erbb2 tumors (p=0.0003). Interestingly, there was a significant negative correlation (Spearman Correlation=−0.31; p=0.02) between nuclear levels of Cathepsin-L and 53BP1 expression in all tumor types.

In summary, the data indicate that activation of CTSL-mediated degradation of 53BP1 is one of the mechanisms responsible for the decrease in 53BP1 in TNBC. Intriguingly, 67% of TN breast tumors exhibited low levels of both 53BP1 and nuclear CTSL, suggesting that alternative mechanisms are activated in TNBC to ensure downregulation of 53BP1. Thus, upregulation of CTSL-mediated degradation of 53BP1 represents a new biomarker of a subset of TNBC which, based in the in vitro studies shown here, could potentially be used as predictor of the response of these specific tumors to DNA damaging therapeutic strategies such as radiation, crosslinking reagents, and PARPi.

TABLE 2
Immunohistochemical results of breast cancer tumors

| | | H score | | | | | |
|---|---|---|---|---|---|---|---|
| | Molecular | Mean | Median | | p- | ≧K* | p- |
| Proteins | Type | (SD) | (IQR) | Range | value | n (%) | value |
| Citoplasmatic | Luminal A | 150(32.0) | 150(52.0) | 70-205 | 0.7118 | 54(77.1) | 0.1161 |
| Cathepsin L | Luminal B | 145(35.9) | 130(47.5) | 75-230 | | 33(71.7) | |
| | Erbb2 | 145(43.9) | 130(73.0) | 80-230 | | 19(54.3) | |
| | Triple | 144(36.7) | 150(55.0) | 80-200 | | 18(66.7) | |
| | Negative | | | | | | |
| Nuclear | Luminal A | 7(14.0) | 0(0.0) | 0-55 | 0.0030 | 1(1.4) | <0.0001 |
| Cathespin L | Luminal B | 8(14.0) | 0(15.0) | 0-55 | | 2(4.3) | |
| | Erbb2 | 11(19.8) | 0(12.5) | 0-75 | | 3(8.6) | |
| | Triple | 38(46.2) | 0(80.0) | 0-150 | | 10(37.0) | |
| | Negative | | | | | | |
| 53BP1 | Luminal A | 152(60.2) | 160(75.0) | 0-300 | 0.0009 | 55(78.6) | 0.0003 |
| | Luminal B | 147(44.6) | 150(48.8) | 40-230 | | 36(80.4) | |
| | Erbb2 | 149(62.6) | 140(82.5) | 20-300 | | 22(68.8) | |
| | Triple | 103(50.0) | 90(65.0) | 0-190 | | 10(35.7) | |
| | Negative | | | | | | |

Proteins expression evaluated by Hscore. Mean (SD = standard deviation) and Median (IQR = Inter quartile range) of Hscore values from immunohistochemical analysis of cytoplasmic and nuclear CTSL and nuclear 53BP1 in the breast cancer subtypes luminal A, luminal B, Erbb2 and Triple-Negative. K represents the cut-off value that maximizes the differences in the expression of an antigen among molecular subtypes. The maximum discriminative cut-off points are 50 for nuclear cathepsin L, and 115 for nuclear 53BP1. n (%) indicates the number and (percentage) of tumors of each molecular subtype with Hscore values equal or above K.

### Example 7

### Nuclear CTSL: a New Predictive Biomarker for Drug Response

The complexity and diversity of cancer phenotypes has fueled the concept of personalized treatment, which utilizes molecular and genetic composition of the specific tumor to design the best therapeutic strategy. For personalized medicine to be effective, it is necessary to identify predictive biomarkers that allow the stratification of patients into different subgroups, and diagnostic tests that determine the clinical response of a patient subgroup to a specific drug. In breast cancers, a small number of predictive biomarkers have been found (La Thangue and Kerr, 2012). For example, tumors with amplification of HER2 or upregulation of estrogen receptor are responsive to drugs that target these receptors. As stated above, BRCA1/2-mutated tumors are responsive to PARPi—olaparib and veliparib—. However, not all tumors in these categories respond in the same manner to the chosen treatments. For example, BRCA1/2-mutated tumors with low expression/levels of 53BP1 are resistant to PARPi. Therefore, these patients will not benefit from this specific treatment unless the levels of 53BP1 are stabilized. Similarly, while TNBC have a poor prognosis in general, there are patients who do well. In contrast, subsets of patients with receptor positive disease have much more aggressive cancer than would be predicted through biomarker analysis. Like TNBC, these receptor positive cancers may benefit from a more aggressive chemotherapy regime early in treatment if they can be identified. Therefore, it is imperative that we identify additional predictive biomarkers for drug response.

### Discussion for Examples 1-7

The present study suggests that nuclear CTSL levels might represent a new predictive biomarker for therapy use in subsets of TNBC patients. In particular, breast cancers with high nuclear CTSL activity and low levels of 53BP1 are likely to be defective in NHEJ, and are probably resistant to PARPi. For these types of tumors, treatment with vitamin D or CTSL inhibitors to stabilize 53BP1 levels in combination with PARPi might result in the most effective therapy. Lastly, the status of this pathway could serve to identify women that could benefit from supplementation with vitamin D as a strategy to reduce malignancy. Breast cancer is the leading cause of cancer death in women worldwide (Jemal et al.). Among all types of breast cancer, Triple-Negative (TN) and BRCA1-deficient tumors are particularly aggressive and difficult to treat. These types of tumors harbor similar DNA repair deficiencies and gene expression profiles (Foulkes et al.). Of particular importance is the loss of BRCA1 function and decrease in 53BP1 levels, two factors with a decisive role in the choice of mechanism of DNA doublestrand breaks (DSBs) repair: homologous recombination (HR) or non-homologous end-joining (NHEJ) (Bouwman et al., 2010). Recent landmark studies demonstrated that loss of 53BP1 allows survival of BRCA1-deficient cells and induces their resistance to DNA damaging therapeutic strategies such as cysplatin, mitomycin C, or PARPi (Bothmer et al., 2010; Bouwman et al., 2010; Bunting et al., 2010; Cao et al., 2009). Thus, stabilization of 53BP1 levels represents a new promising strategy for the treatment of these types of cancers. However, prior to this study, no information was available about how the levels of 53BP1 are regulated in breast tumor cells. Here, we demonstrate a novel function for BRCA1 in the regulation of 53BP1 protein levels. We show that acute depletion of BRCA1 leads to activation of CTSL-mediated degradation of 53BP1 which in turn rescues defects in HR, genomic stability, and proliferation/viability of BRCA1-deficient cells. Accordingly, depletion of CTSL or inhibition of its activity stabilizes 53BP1 protein levels and induces genomic instability in the context of BRCA1 deficiency. Furthermore, we show a significant correlation between high levels of nuclear CTSL, low levels of 53BP1, and the Triple-Negative breast cancer phenotype. This study has revealed a new pathway that is activated upon loss of BRCA1 function that is expected to contribute to the progression of breast cancers of the poorest prognosis. Inhibition of this pathway by treatment with vitamin D or cathepsin inhibitors could provide a new therapeutic strategy for breast cancer. Importantly, the status of the pathway offers great potential as a predictive biomarker for response to drugs that induce DNA damage. In addition, our data indicate that the levels of 53BP1 and BRCA1 are regulated throughout the cell cycle in order to maintain an active competition between repair pathways that is important to maintain genome integrity. Tilting the balance of DNA DSBs repair toward one specific pathway by loss of either BRCA1 or 53BP1 function could be exploited for therapeutic purposes.

CTSL: a New Target of BRCA1 with an Important Function in DNA Repair

CTSL is one of the most abundant proteases in the endosomal/lysosomal compartment and has also been identified in the nucleus (Duncan et al., 2008; Goulet et al., 2004). The nuclear form of CTSL arises from the initiation of translation at downstream AUG sites that spare the sequence coding for the signal peptide that targets the protein to the ER. Upregulation of CTSL is a hallmark of a variety of cancers and has been correlated with increased invasiveness, metastasis, and overall degree of malignancy (Gocheva and Joyce, 2007; Jedeszko and Sloane, 2004; Skrzydlewska et al., 2005). Thus, inhibition of CTSL activity is considered a promising strategy for cancer treatment (Lankelma et al.). In addition to the previously reported effects of CTSL upregulation on the degradation of extracellular matrix components and cell-adhesion molecules, studies in our laboratory showed that CTSL upregulation leads to accumulation of the protease in the nucleus and degradation of nuclear factors with key roles in cell cycle regulation—Rb family members—and DNA repair—53BP1-. As a consequence, upregulation of CTSL leads to defects in DNA DSBs repair (Gonzalez-Suarez et al., 2011). The present study demonstrates that loss of BRCA1 is one of the mechanisms leading to transcriptional upregulation of CTSL, and most importantly, that CTSL-mediated degradation of 53BP1 allows BRCA1-deficient cells to overcome genomic instability and ensure survival.

A number of molecular mechanisms and signaling pathways have been implicated in the regulation of CTSL expression. For example, pro-inflammatory cytokines, oncogenes, and tumor promoters, such as Stat3 and oncogenic Ras, increase CTSL expression (Collette et al., 2004; Kreuzaler et al., 2011). Ras, in particular, leads to the upregulation of the nuclear form of CTSL, promoting proliferation of cancer cells (Goulet et al., 2007). In addition, CTSL is upregulated epigenetically by promoter hypomethylation in some tumor cells (Jean et al., 2008; Samaiya et al., 2010). A whole body of evidence indicates that BRCA1 can act as a coactivator or corepressor of transcription (Mullan et al., 2006). Interestingly, the DNA methyltransferase DNMT1 is regulated transcriptionally by BRCA1 (Shukla et al.). Specifically, BRCA1 maintains the DNMT1 gene promoter in a transcriptionally active configuration and loss of BRCA1 leads to global DNA hypomethylation and increased expression of several proto-oncogenes. Thus, the loss of BRCA1 might promote transcription of CTSL by promoter hypomethylation. Future studies need to determine if the CTSL gene promoter is subjected to epigenetic regulation upon loss of BRCA1, or if alternatively, BRCA1-deficiency increases promoter activity by facilitating the binding of transcriptional activators or by decreasing the binding of transcriptional repressors.

### Functional Relationship Between CTSL, 53BP1 and BRCA1 Levels During the Cell Cycle

A whole body of evidence indicates that NHEJ is active in all phases of the cell cycle while HR is particularly important in late S/G2, where sister chromatids are available to be used as substrates for recombination (Rothkamm et al., 2003). Accordingly, BRCA1 levels fluctuate during the cell cycle and markedly increase during S/G2 phases (Gudas et al., 1996). The levels of 53BP1 during the cell cycle have not been previously reported. However, given that NHEJ is the primary form of repair of DSBs during G1, it has been traditionally thought that 53BP1 must be in excess during this phase of the cycle. Surprisingly, we find that the levels of 53BP1 mirror those of BRCA1 throughout the cycle. These results suggest that the levels of both proteins are co-regulated in order to maintain proper competition for DNA DSBs processing by HR and NHEJ. Excess of either protein at a specific phase of the cycle might result in genomic instability, as shown in cells deficient in either 53BP1 or BRCA1.

The inverse correlation between CSTL levels and those of BRCA1 and 53BP1 during the cell cycle suggests a potential functional relationship between these proteins. We envisioned two possible scenarios: (i) the decrease in BRCA1 levels in specific phases of the cell cycle is independent of CTSL, but activates CTSL-mediated degradation of 53BP1; (ii) upregulation of CTSL leads to degradation of both BRCA1 and 53BP1 proteins. The present study clearly shows that CTSL contributes to the decreased levels of 53BP1 and BRCA1 in G0/G1 arrested cells, since depletion or inhibition of CTSL stabilizes the levels of both proteins in growth-arrested cells. This is supported by previous studies showing that BRCA1 itself is a target for degradation by cysteine proteases of the cathepsin family, although the specific cathepsin was not identified (Blagosklonny et al., 1999). We propose a model where the decrease in BRCA1 levels in G0/G1 phases of the cell cycle activates transcription of CTSL and degradation of 53BP1. Upregulation of CTSL would also trigger a feedback mechanism that lowers the levels of BRCA1 protein as well. At later stages of the cell cycle, the upregulation of BRCA1 and downregulation of CTSL would stabilize the levels of 53BP1. Overall, our findings support an important role for CTSL not only in the regulation of 53BP1 stability in tumor cells, but also as a regulator of protein stability during the cell cycle in normal cells. This novel role of CTSL would ensure the balance between BRCA1 and 53BP1 levels and the competition between repair pathways throughout the cell cycle, contributing to safeguard the integrity of the genome.

### Vitamin D and Cathepsin Inhibitors can Modulate DNA DSBs Repair Choice

Our previous studies in mouse cells and the present study in breast cancer cells have revealed an unprecedented role for vitamin D and cathepsin inhibitors in the regulation of DNA DSBs repair choice. By stabilizing 53BP1 protein levels in the context of BRCA1 deficiency, these compounds facilitate repair of DSBs by NHEJ while inhibiting HR. Importantly, our data indicate that both vitamin D and cathepsin inhibitors impact on 53BP1 stability especially in cells that upregulate CTSL, i.e. BRCA1-deficient cells, showing a lesser effect in cells with normal CTSL expression. This is likely due to the fact that upregulation of CTSL leads to an increase in the levels of nuclear CTSL, which is low relative to other cellular compartments in normal cells (Gonzalez-Suarez et al., 2011). Thus, although upregulation of CTSL leads to an increase in the levels of the protease at all cellular locations, as well as in the extracellular matrix, the nuclear form of the protease would be responsible for the degradation of 53BP1.

The ability to impact the choice of DNA DSBs repair could have profound consequences for cancer therapy. In tumor cells that downregulate 53BP1 as a means to ensure proliferation and viability, BRCA1 deficient and Triple-Negative, treatment with vitamin D can stabilize 53BP1, increase NHEJ-mediated genomic instability, and induce growth arrest and/or cell death. Thus, treatment with vitamin D or CTSL inhibitors represents a promising therapeutic strategy for breast tumors of the poorest prognosis. A whole body of evidence also indicates that vitamin D supplementation and the activation of the vitamin D receptor (VDR) signaling pathway protect from malignant transformation. This effect could be mediated in part by the ability of vitamin D to attenuate DNA damage levels via induction of expression of DNA repair genes such as ATM and RAD50 (Ting et al., 2012). Interestingly, vitamin D also upregulates VDR-mediated transcription of BRCA1 (Campbell et al., 2000). Thus, vitamin D could have a dual beneficial effect on cancer by preventing the genomic instability that drives malignant transformation as well as by modulating the choice of DNA repair pathways in full grown tumors, which could be exploited with therapeutic purposes. In BRCA1-mutation carriers for example, supplementation with vitamin D might maintain low levels of CTSL and therefore prevent the downregulation of 53BP1 that is associated with the progression of the tumors, possibly delaying cancer onset in this population. Once the tumors develop in this population, vitamin D treatment could be used to induce NHEJ-mediated genomic instability and cell death. Thus, treatment with vitamin D could represent a safe and cost-efficient therapeutic strategy for specific types of breast cancer.

A major breakthrough in the treatment of BRCA1-mutated and TNBC was the finding that these tumors are exquisitely sensitive to PARPi (Farmer et al., 2005; Helleday et al., 2005). Depletion of PARP1 by siRNA or inhibition of its activity by a number of compounds—KU0058684, KU0058948, and AG014699—caused a clear reduction in clonogenic survival of BRCA1-deficient cells when compared to cells expressing BRCA1 (Drew et al., 2010; Farmer et al., 2005; Helleday et al., 2005). In addition, acute depletion of BRCA1 in MCF7 cells increased profoundly their sensitivity to PARPi (Farmer et al., 2005). Inhibition of PARP activity hinders single-strand breaks (SSBs) repair, which causes stalling of the replication fork and formation of DSBs that need to be repaired primarily by HR, an error free repair mechanism (Ashworth, 2008; Helleday et al., 2005). BRCA1-deficient cells cannot deal with the amount of DSBs generated by PARPi, resulting in proliferation arrest and cell death. The demonstrated vulnerability of BRCA1 deficient cells to PARPi expedited their use in the clinic (Fong et al., 2009). Phase II studies with PARPi have shown a significant response rate in women carrying BRCA1 mutations and in sporadic TNBC, with tolerable side effects (Tutt et al., 2010). Thus, the use of PARPi as single agents or in combination with radiation or chemotherapy is a leading strategy for breast cancer management, especially for HR deficient tumors. However, a significant fraction of these cancers acquire resistance to PARPi underscoring the importance of understanding the molecular mechanisms behind resistance to design more effective therapeutic strategies. Importantly, recent studies demonstrated that loss of 53BP1 reduces the sensitivity of BRCA1-deficient cells to PARPi by rescuing the ability of these cells to repair DNA DSBs by HR (Bunting et al., 2010). Our study suggests that inhibition of CTSL activity would increase the sensitivity to PARPi of BRCA1-deficient cells that downregulate 53BP1 levels as a mean to ensure proliferation and viability, by hindering the ability of these cells to lower 53BP1 levels.

### REFERENCES

Aly, A., and Ganesan, S. (2011). “BRCA1, PARP, and 53BP1: conditional synthetic lethality and synthetic viability.” J Mol Cell Biol 3, 66-74.
Anderson, S. F. et al. (1998). “BRCA1 protein is linked to the RNA polymerase II holoenzyme complex via RNA helicase A.” Nat Genet. 19: 254-256.
Ashworth, A. (2008). “A synthetic lethal therapeutic approach: poly(ADP) ribose polymerase inhibitors for the treatment of cancers deficient in DNA double-strand break repair.” J Clin Oncol 26: 3785-3790.
Blagosklonny, M. V. et al. (1999). “Regulation of BRCA1 by protein degradation.” Oncogene 18, 6460-6468.
Bochar, D. A. et al. (2000). “BRCA1 is associated with a human SWI/SNF-related complex: linking chromatin remodeling to breast cancer.” Cell 102: 257-265.
Bothmer, A. et al. (2010). “53BP1 regulates DNA resection and the choice between classical and alternative end joining during class switch recombination.” J Exp Med 207, 855-865.
Bouwman, P. et al. (2010). “53BP1 loss rescues BRCA1 deficiency and is associated with triple-negative and BRCA-mutated breast cancers.” Nat Struct Mol Biol 17: 688-695.
Bryant, H. E. et al. (2005). “Specific killing of BRCA2-deficient tumours with inhibitors of poly(ADP-ribose) polymerase.” Nature 434: 913-917.
Bunting, S. F. et al. (2010). “53BP1 inhibits homologous recombination in Brca1-deficient cells by blocking resection of DNA breaks.” Cell 141: 243-254.
Campbell, M. J. et al. (2000). “The anti-proliferative effects of 1alpha,25(OH)2D3 on breast and prostate cancer cells are associated with induction of BRCA1 gene expression.” Oncogene 19, 5091-5097.
Cao, L. et al. (2006). “ATM-Chk2-p53 activation prevents tumorigenesis at an expense of organ homeostasis upon Brca1 deficiency.” Embo J 25: 2167-2177.
Cao, L. et al. (2009). “A selective requirement for 53BP1 in the biological response to genomic instability induced by Brca1 deficiency.” Mol Cell 35: 534-541.
Collette, J. et al. (2004). “Enhanced cathepsin L expression is mediated by different Ras effector pathways in fibroblasts and epithelial cells.” Int J Cancer 112: 190-199.
Difilippantonio, S. et al. (2008). “53BP1 facilitates long-range DNA end-joining during V(D)J recombination.” Nature 456: 529-533.
Dimitrova, N. et al. (2008). “53BP1 promotes non-homologous end joining of telomeres by increasing chromatin mobility.” Nature 456: 524-528.
Drew, Y. et al. (2010). “Therapeutic potential of poly(ADP-ribose) polymerase inhibitor AG014699 in human cancers with mutated or methylated BRCA1 or BRCA2.” J Natl Cancer Inst 103: 334-346.
Duncan, E. M. et al. (2008). “Cathepsin L proteolytically processes histone H3 during mouse embryonic stem cell differentiation.” Cell 135: 284-294.
Evers, B., and Jonkers, J. (2006). “Mouse models of BRCA1 and BRCA2 deficiency: past lessons, current understanding and future prospects.” Oncogene 25: 5885-5897.
Fan, S. et al. (1999). “BRCA1 inhibition of estrogen receptor signaling in transfected cells.” Science 284: 1354-1356.
Farmer, H. et al. (2005). “Targeting the DNA repair defect in BRCA mutant cells as a therapeutic strategy.” Nature 434: 917-921.
Fernandez-Capetillo, O. et al. (2002). “DNA damage-induced G2-M checkpoint activation by histone H2AX and 53BP1.” Nat Cell Biol 4: 993-997.
Fong, P. C., et al. (2009). “Inhibition of poly(ADP-ribose) polymerase in tumors from BRCA mutation carriers.” N Engl J Med 361: 123-134.
Foulkes, W. D., Smith, I. E., and Reis-Filho, J. S. (2010). “Triple-negative breast cancer.” N Engl J Med 363: 1938-1948.
Gartner, E. M., Burger, A. M., and Lorusso, P. M. (2010). “Poly(adp-ribose) polymerase inhibitors: a novel drug class with a promising future.” Cancer J 16: 83-90.
Gocheva, V., and Joyce, J. A. (2007). “Cysteine cathepsins and the cutting edge of cancer invasion.” Cell Cycle 6: 60-64.
Gonzalez-Suarez, I. et al. (2011). “A new pathway that regulates 53BP1 stability implicates Cathepsin L and vitamin D in DNA repair.” Embo J 30: 3383-3396.
Gonzalez-Suarez, I. et al. (2009). “Novel roles for A-type lamins in telomere biology and the DNA damage response pathway.” Embo J 28: 2414-2427.
Goulet, B. et al. (2004). “A cathepsin L isoform that is devoid of a signal peptide localizes to the nucleus in S phase and processes the CDP/Cux transcription factor.” Mol Cell 14: 207-219.
Goulet, B., et al. (2007). “Increased expression and activity of nuclear cathepsin L in cancer cells suggests a novel mechanism of cell transformation.” Mol Cancer Res 5: 899-907.
Gudas, J. M. Liet al. (1996). “Cell cycle regulation of BRCA1 messenger RNA in human breast epithelial cells.” Cell Growth Differ 7: 717-723.
Helleday, T. et al. (2005). “Poly(ADP-ribose) polymerase (PARP-1) in homologous recombination and as a target for cancer therapy.” Cell Cycle 4: 1176-1178.
Iliakis, G. et al. (2004). “Mechanisms of DNA double strand break repair and chromosome aberration formation.” Cytogenet Genome Res 104: 14-20.
Jean, D. et al. (2008). “Cathepsin L expression is up-regulated by hypoxia in human melanoma cells: role of its 5′-untranslated region.” Biochem J 413, 125-134.
Jedeszko, C., and Sloane, B. F. (2004). “Cysteine cathepsins in human cancer.” Biol Chem 385: 1017-1027.
Jemal, A. et al. (2010). “Global patterns of cancer incidence and mortality rates and trends.” Cancer Epidemiol Biomarkers Prev 19: 1893-1907.
Kreuzaler, P. A. et al. (2011). “Stat3 controls lysosomal-mediated cell death in vivo.” Nat Cell Biol 13: 303-309.
La Thangue, N. B., and Kerr, D. J. (2012). “Predictive biomarkers: a paradigm shift towards personalized cancer medicine.” Nature reviews 8: 587-596.
Lankelma, J. M. et al. (2010). <<Cathepsin L, target in cancer treatment?” Life Sci 86: 225-233.
Moynahan, M. E. et al. (1999). “Brca1 controls homology-directed DNA repair.” Mol Cell 4: 511-518.
Moynahan, M. E., Cui, T. Y., and Jasin, M. (2001). “Homology-directed dna repair, mitomycin-c resistance, and chromosome stability is restored with correction of a Brca1 mutation.” Cancer research 61: 4842-4850.
Mullan, P. B., Quinn, J. E., and Harkin, D. P. (2006). “The role of BRCA1 in transcriptional regulation and cell cycle control.” Oncogene 25: 5854-5863.
Neuhausen, S. L., and Marshall, C. J. (1994). “Loss of heterozygosity in familial tumors from three BRCA1-linked kindreds.” Cancer research 54: 6069-6072.
Olive, P. L et al. (1990). “Heterogeneity in radiation-induced DNA damage and repair in tumor and normal cells measured using the “comet” assay.” Radiat Res 122, 86-94.
Ouchi, T., et al. (1998). “BRCA1 regulates p53-dependent gene expression.” PNAS. 95: 2302-2306.
Pallares, J., et al. (2009). “A review of the applications of tissue microarray technology in understanding the molecular features of endometrial carcinoma.” Analytical and quantitative cytology and histology/the International Academy of Cytology [and] American Society of Cytology 31: 217-226.
Redwood, A. B. et al. (2011). “A dual role for A-type lamins in DNA double-strand break repair. Cell Cycle 10: 2549-2560.
Rothkamm, K. et al. (2003). “Pathways of DNA double-strand break repair during the mammalian cell cycle.” Mol Cell Biol 23: 5706-5715.
Samaiya, M. et al. (2010). “Epigenetic Regulation of Cathepsin L Expression in Chronic Myeloid Leukemia.” J Cell Mol. Med.
Schultz, L. B. et al. (2000). “p53 binding protein 1 (53BP1) is an early participant in the cellular response to DNA double-strand breaks.” J Cell Biol 151: 1381-1390.
Scully, R. et al. (1997). “Dynamic changes of BRCA1 subnuclear location and phosphorylation state are initiated by DNA damage.” Cell 90: 425-435.
Scully, R. et al. (1996). “Location of BRCA1 in human breast and ovarian cancer cells.” Science 272: 123-126.
Scully, R., and Livingston, D. M. (2000). “In search of the tumour-suppressor functions of BRCA1 and BRCA2.” Nature 408: 429-432.
Shakya, R. et al. (2008). “The basal-like mammary carcinomas induced by Brca1 or Bard1 inactivation implicate the BRCA1/BARD1 heterodimer in tumor suppression. PNAS. 105: 7040-7045.
Shukla, V. et al. (2010). “BRCA1 affects global DNA methylation through regulation of DNMT1.” Cell Res 20: 1201-1215.
Skrzydlewska, E. et al. (2005). “Proteolytic-antiproteolytic balance and its regulation in carcinogenesis.” World J Gastroenterol 11: 1251-1266.
Snouwaert, J. N. et al (1999). “BRCA1 deficient embryonic stem cells display a decreased homologous recombination frequency and an increased frequency of nonhomologous recombination that is corrected by expression of a brca1 transgene.” Oncogene 18: 7900-7907.
Sorlie, T. et al. (2003). “Repeated observation of breast tumor subtypes in independent gene expression data sets.” Proceedings of the National Academy of Sciences of the United States of America. 100: 8418-8423.
Ting, H. J. et al. (2012). “A Positive Feedback Signaling Loop between ATM and the Vitamin D Receptor Is Critical for Cancer Chemoprevention by Vitamin D.” Cancer research.
Turner, N. C. et al. (2006). “Basal-like breast cancer and the BRCA1 phenotype.” Oncogene 25: 5846-5853.
Tutt, A. et al. (2010). “Oral poly(ADP-ribose) polymerase inhibitor olaparib in patients with BRCA1 or BRCA2 mutations and advanced breast cancer: a proof-of-concept trial.” Lancet 376: 235-244.
Vaughn, J. P. et al. (1996). “BRCA1 expression is induced before DNA synthesis in both normal and tumor-derived breast cells.” Cell Growth Differ 7: 711-715.
Wang, B. et al. (2002). “53BP1, a mediator of the DNA damage checkpoint.” Science 298: 1435-1438.
Wang, Q. et al. (1998). “BRCA1 binds c-Myc and inhibits its transcriptional and transforming activity in cells.” Oncogene 17: 1939-1948.
Ward, I. M. et al. (2003). “p53 Binding protein 53BP1 is required for DNA damage responses and tumor suppression in mice.” Mol Cell Biol 23: 2556-2563.
Westermark, U. K. et al. (2003). “BARD1 participates with BRCA1 in homology-directed repair of chromosome breaks.” Mol Cell Biol 23: 7926-7936.
Wooster, R., and Weber, B. L. (2003). “Breast and ovarian cancer.” N Engl J Med 348: 2339-2347.
Xie, A. et al. (2007). “Distinct roles of chromatin-associated proteins MDC1 and 53BP1 in mammalian double-strand break repair.” Mol Cell 28: 1045-1057.
Xu, B., Kim, S., and Kastan, M. B. (2001). “Involvement of Brca1 in S-phase and G(2)-phase checkpoints after ionizing irradiation.” Mol Cell Biol 21: 3445-3450.
Xu, X. et al. (1999). “Centrosome amplification and a defective G2-M cell cycle checkpoint induce genetic instability in BRCA1 exon 11 isoform-deficient cells.” Mol Cell 3: 389-395.
Zhu, Q., et al. (2011). “BRCA1 tumour suppression occurs via heterochromatin-mediated silencing.” Nature 477: 179-184.

What is claimed is:

1. A method for classifying a breast tumor, the method comprising:
(a) obtaining a sample of a breast tumor;
(b) processing the sample in vitro to determine the levels of nuclear cathepsin L and nuclear 53BP1 in at least one tumor cell comprising the sample; and
(c) classifying the tumor as having (i) high nuclear cathepsin L and low nuclear 53BP1, (ii) high nuclear cathepsin L and high nuclear 53BP1, or (ii) low nuclear cathepsin L and low nuclear 53BP1.

2. The method of claim 1, wherein the method of processing the sample in vitro to determine the levels of nuclear cathepsin L and nuclear 53BP1 is selected from the group consisting of immunohistochemistry, flow cytometry, array and ELISA.

3. The method of claim 1, wherein the breast tumor is selected from the group consisting of basal-like tumors, triple negative breast tumors, BRCA1-deficient tumors, and combinations thereof.

4. The method of claim 3, wherein high and low nuclear cathepsin L and nuclear 53BP1 are determined from the average level of nuclear CTSL and nuclear 53BP1 protein expression in a population of breast tumors, wherein a high level is above the average and a low level is below the average.

5. The method of claim 1, wherein the method of processing the sample in vitro to determine the levels of nuclear cathepsin L and nuclear 53BP1 is immunohistochemistry.

6. The method of claim 5, wherein an Hscore value or no greater than about 50 is used to discriminate a high levels of nuclear cathepsin L from a low levels of nuclear cathepsin L, and an Hscore value of no less than about 115 is used to discriminate high levels of nuclear 53BP1 from low levels of nuclear 53BP1.

7. A method for increasing the sensitivity of a tumor cell to a DNA-damaging agent, the method comprising contacting the tumor cell with an effective amount of a cathepsin L inhibitor and a DNA-damaging agent.

8. The method of claim 7, wherein the tumor cell is in a breast tumor in a subject.

9. The method of claim 8, wherein the breast tumor is selected from the group consisting of basal-like tumors, triple negative breast tumors, BRCA1-deficient tumors, and combinations thereof.

10. The method of claim 7, wherein the cathepsin L inhibitor is vitamin D.

11. The method of claim 7, wherein the DNA-damaging agent is a PARP inhibitor.

12. The method of claim 7, wherein the DNA-damaging agent is radiation.

13. A method for predicting the effectiveness of a DNA damaging agent in reducing tumor growth in a subject in need thereof, the method comprising:
(a) obtaining a sample of a breast tumor from the subject;
(b) processing the sample in vitro to determine the levels of nuclear CTSL and nuclear 53BP1 in at least one tumor cell comprising the sample;
(c) classifying the tumor as non-responsive to a DNA-damaging agents if the sample has (i) high nuclear CTSL and low nuclear 53BP1 or (ii) low nuclear CTSL and low nuclear 53BP1; and
(d) identifying a subject with a tumor classified as non-responsive to a DNA-damaging agent as a subject for which a DNA-damaging agent would not be effective.

14. The method of claim 13, wherein the breast tumor is selected from the group consisting of basal-like tumors, triple negative breast tumors, BRCA1-deficient tumors, and combinations thereof.

15. The method of claim 14, wherein high and low nuclear cathepsin L and nuclear 53BP1 are determined from the average level of nuclear CTSL and nuclear 53BP1 protein expression in a population of breast tumors, wherein a high level is above the average and a low level is below the average.

16. The method of claim 13, wherein the DNA damaging agent is selected from the group consisting of PARP inhibitors and radiation.

17. The method of claim 13, wherein the method of processing the sample in vitro to determine the levels of nuclear cathepsin L and nuclear 53BP1 is selected from the group consisting of immunohistochemistry, flow cytometry, array and ELISA.

18. The method of claim 13, wherein the method of processing the sample in vitro to determine the levels of nuclear cathepsin L and nuclear 53BP1 is immunohistochemistry.

19. The method of claim 13, wherein an Hscore value of no greater than about 50 is used to discriminate a high level of nuclear cathepsin L from a low level of nuclear cathepsin L, and an Hscore value of no less than about 115 is used to discriminate a high level of nuclear 53BP1 from a low level of nuclear 53BP1.
